(19) United States
(12) Patent Application Publication
(10) Pub. No.: US 2005/0288333 A1
(43) Pub. Date: Dec. 29, 2005

(54) Controlling angiogenesis with anabaseine analogs

(76) Inventor: William R. Kem, Gainesville, FL (US)

Correspondence Address: AKERMAN SENTERFITT, P.O. BOX 3188, WEST PALM BEACH, FL, 33402-3188, US

(21) Appl. No.: 11/147,996

(22) Filed: Jun. 8, 2005

Related U.S. Application Data
(60) Provisional application No. 60/577,990, filed on Jun. 8, 2004.

(51) Int. Cl. A61K 31/444

(52) U.S. Cl. 514/332

(57) ABSTRACT

Compounds controlling angiogenesis and vasculogenesis. In particular, induction of angiogenesis to promote growth of new vasculature by the use of anabaseine agonists and to the reduction of pathological angiogenesis by the use of anabaseine antagonists.

### CROSS-REFERENCE TO RELATED APPLICATIONS

The present application claims the priority of U.S. provisional patent application No. 60/577,990, entitled “CONTROLLING ANGIOGENESIS WITH ANABASEINE ANALOGS,” filed Jun. 8, 2004. The foregoing is incorporated herein by reference in its entirety.

### STATEMENT AS TO FEDERALLY SPONSORED RESEARCH

The US, government owns rights in the present invention pursuant to grant numbers R01-MH6142 from the United States National Institutes of Health.

### FIELD OF THE INVENTION

The invention relates generally to the field of controlling angiogenesis and vasculogenesis, particularly to the induction of angiogenesis to promote growth of new vasculature by the use of anabaseine agonists and to the reduction of pathological angiogenesis by the use of anabaseine antagonists.

### BACKGROUND

Angiogenesis and vasculogenesis are processes involved in the growth of blood vessels. Angiogenesis is the process by which new blood vessels are formed from extant capillaries, while vasculogenesis involves the growth of vessels deriving from endothelial progenitor cells. Angiogenesis is a complex, combinatorial process that is regulated by a balance between pro- and anti-angiogenic molecules. Angiogenic stimuli (e.g. hypoxia or inflammatory cytokines) result in the induced expression and release of angiogenic growth factors such as vascular endothelial growth factor (VEGF) or fibroblast growth factor (FGF). These growth factors stimulate endothelial cells (EC) in the existing vasculature to proliferate and migrate through the tissue to form new endothelialized channels.

Angiogenesis and vasculogenesis, and the factors that regulate these processes, are important in embryonic development, inflammation, and wound healing, and also contribute to pathologic conditions such as tumor growth, diabetic retinopathy, rheumatoid arthritis, and chronic inflammatory diseases (see, e.g., U.S. Pat. No. 5,318,957; Yancopoulos et al., Cell 1998, 93:661-4; Folkman et al, Cell, 1996, 87:1153-1155; and Hanahan et al., Cell, 1996, 86:353-64).

Both angiogenesis and vasculogenesis involve the proliferation of endothelial cells. Endothelial cells line the walls of blood vessels; capillaries are comprised almost entirely of endothelial cells. The angiogenic process involves not only increased endothelial cell proliferation but also comprises a cascade of additional events, including protease secretion by endothielial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiation into endothelial cells which then from de novo vessels (see, e.g., Folkman et al., Cell, 1996, 87:1153-1155).

Several angiogenic and/or vasculogenic agents with different properties and mechanisms of action have been described. For example, acidic and basic fibroblast growth factor (FGF), transforming growth factor alpha (TGF-α) and beta (TGF-β), tumor necrosis factor (TNF), platelet-derived growth factor (PDGF), vascular endothelial cell growth factor (VEGF), and angiogenin are potent and well-characterized angiogenesis promoting agents. In addition, both nitric oxide and prostaglandin (a prostacyclin agonist) have been shown to be mediators of various angiogenic growth factors, such as VEGF and bFGF. However, the therapeutic applicability of some of these compounds, especially as systemic agents, is limited by their potent tendency to induce growth of unwanted deformities.

Angiogenesis and vasculogenesis have been the focus of intense interest since these processes can be exploited to therapeutic advantage. Stimulation of angiogenesis and/or vasculogenesis can aid in the healing of wounds, the vascularizing of skin grafts, and the enhancement of collateral circulation where there has been vascular occlusion or stenosis (e.g., to develop a ‘biobypass” around an obstruction due to coronary, carotid, or peripheral arterial occlusion disease). There is a need for methods that are well-tolerated by the subject, but that are of high efficacy in effecting stimulation of angiogenesis and/or vasculogenesis.

On the other hand, inappropriate, or pathological, angiogenesis is involved in the growth of atherosclerotic plaque, diabetic retinopathy, degenerative maculopathy, retrolental fibroplasia, idiopathic pulmonary fibrosis, acute adult respiratory distress syndrome, and asthma. Furthermore, tumor progression is associated with neovascularization, which provides a mechanism by which nutrients are delivered to the progressively growing tumor tissue. Thus, there is also a need for methods of reducing pathological angiogenesis. The present invention addresses these needs.

### SUMMARY

The present invention provides methods for controlling angiogenesis in a mammal by administration of anabaseine agonists or anabaseine antagonists. These methods provide treatment for pathological conditions involving angiogenesis.

In a preferred embodiment, the anabaseine compounds comprise:

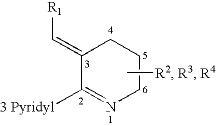

or a pharmaceutically acceptable salt thereof, wherein R¹ is hydrogen methyl, propyl, ethyl, acetoxy, acetamido, amino, dimethylcarbamate, dimethylaminopropoxy, hydroxyl, methoxy, isopropoxy, trifluromethoxy or thiomethoxy or C₁-C₄ alkyl; and R² is hydrogen, methyl, propyl, ethyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or,

wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro;

wherein the compound functions as either an anabaseine agonist or an anabaseine antagonist.

In a preferred embodiment, R² at position 4, R³ at position 5 and R⁴ at position 6 on the terahydropyridyl ring are methyl groups either singly substituted, for example, R² is a methyl group or each is substituted with methyl and single substitutions are in (S)⁻ or (R)⁻ (alpha or beta) enantiomeric form.

In another preferred embodiment, R² at position 4, R³ at position 5 and R⁴ at position 6 on the terahydropyridyl ring are substituted with one or more of: methyl, propyl, ethyl groups.

One embodiment of the present invention involves methods for stimulation of angiogenesis in a mammal by administration of an anabaseine agonist. Stimulation or induction of angiogenesis by the methods of the invention can be used in therapeutic angiogenesis in, for example, treatment of ischemic syndromes such as coronary or peripheral arterial disease.

Another embodiment of the present invention provides a method of treating and preventing diseases and ailments involving angiogenesis such as myocardial and cerebral infarctions, mesenteric or limb ischemia, wounds, and vascular occlusion or stenosis.

A further embodiment of this invention is to provide a method of enhancing angiogenesis to accelerate wound healing, or the vascularization of a skin graft, musculocutaneous flap or other surgically transplanted tissue; or to enhance the healing of a surgically created anastomosis.

The methods generally involve administering an anabaseine agonist preferably an α7-nAChR anabaseine agonist, in an amount effective to induce angiogenesis. The anabaseine agonist can be administered by any route of administration, including, but not limited to, intravenous intra-arterial, intra-pericardial, intramuscular, by inhalation, transdermal, systemic or local, in or around a wound, and topical.

A further embodiment provides a method of treating a disorder associated with pathological angiogenesis. In some embodiments, the invention provides a method of inhibiting abnormal fibrovascular growth. In some of these embodiments, the abnormal fibrovascular growth is associated with inflammatory arthritis. In some embodiments, the invention provides a method of inhibiting a proliferative retinopathy. In some of these embodiments, the proliferative retinopathy occurs as a result of diabetes. The methods generally involve administering anabaseine antagonist, preferably an α7-nAChR anabaseine antagonist, in an amount effective to reduce pathological angiogenesis. In some embodiments, the methods further comprise administering a second angiogenesis inhibitor.

The present invention further provides a method of inhibiting tumor growth. In some embodiments, the invention features a method of inhibiting pathological neovascularization associated with a tumor. The methods generally involve administering an anabaseine antagonist, preferably an α7-nAChR anabaseine antagonist, in an amount effective to reduce angiogenesis associated with a tumor. In some embodiments, the invention further comprises administering an anti-tumor chemotherapeutic agent other than an anabaseine antagonist.

The anabaseine antagonist can be administered by any route of administration, including, but not limited to, intravenous, in or around a solid tumor, systemic, intraarterial, and topical.

Suitable anabaseine agonists and anabaseine antagonists for use in the methods of the invention include, but are not limited to, compounds of the formula:

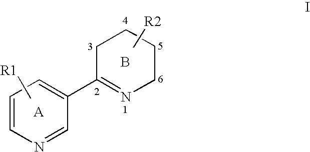

or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, propyl, ethyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or

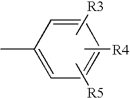

wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is

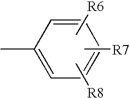

wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; wherein the compound of Formula I and/or II functions as either an anabaseine agonist or an anabaseine antagonist. In the embodiments of the invention involving methods for induction of angiogenesis in a mammal, the compounds of Formula I and/or II that function as anabaseine agonists are administered. In the embodiments of the invention involving methods of reducing angiogenesis in a mammal, the compounds of Formula I and/or II that function as anabaseine antagonists are administered.

In another preferred embodiment, the R¹⁻⁶ are in (S)⁻ or (R)⁻ alpha or beta enantiomeric form. Preferably R² at position 4, R³ at position 5 and R⁴ at position 6 on the terahydropyridyl ring are in (S)⁻ or (R)⁻ alpha or beta enantiomeric form.

In a preferred embodiment, anabaseine agonists and anabaseine antagonists for use in the methods of the invention include, but are not limited to, compounds of the formula:

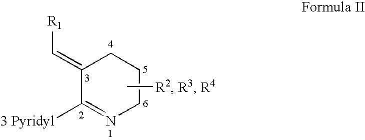

or a pharmaceutically acceptable salt thereof; wherein R¹ is hydrogen, methyl, propyl, ethyl, acetoxy, acetamido, amino, dimethylcarbamate, dimethylaminopropoxy, hydroxyl, methoxy, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or,

wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro.

In a preferred embodiment, R² at position 4, R³ at position 5 and R⁴ at position 6 on the terahydropyridyl ring are methyl groups either singly substituted, for example, R² is a methyl group or each is substituted with methyl and single substitutions are in (S)⁻ or (R)⁻ (alpha or beta) enantiomeric form.

In another preferred embodiment, R² at position 4, R³ at position 5 and R⁴ at position 6 on the terahydropyridyl ring are substituted with one or more of: methyl, propyl, ethyl groups.

The present invention shows that selection of appropriate substituents on the tetrahydropyridyl and pyridyl ring portions of anabaseine compounds determines alpha7 selectivity, either when done separately or in combinations. Certain substituents also determine alpha7 receptor efficacy: Some substituents increase efficacy over benzylidene-anabaseines such as DMXBA while other reduce efficacy to essentially zero, thereby creating a new group of alpha7 nAChR antagonists.

Possible applications of these new alpha7 agonists and antagonists based on the anabaseine structure include therapeutic treatments for neurodegenerative diseases and nicotinic receptor involved addictions as well as potential for development as antiproliferation drugs. In particular, it is shown that altering anabaseine compound polarity and ionization can permit drug application and localization to the peripheral (blood and interstitial fluid) compartments without significant entry into the central nervous system.

In addition to CNS applications, this invention is expected to provide therapeutic agents that selectively stimulate peripheral alpha7 receptors expressed on non-neuronal cells such as macrophages, vascular endothelium and bronchial epithelium, which are peripheral cells known to express functional alpha7 nAChRs. When macrophage alpha7 receptors are stimulated, the secretion of inflammatory cytokines such as TNF is inhibited. These cytokines are known to exacerbate an immune response when overproduced and not efficiently removed from the system. Stimulation of vascular endothelial cells, for example, is known to enhance angiogenesis.

An important aspect of the invention is the expectation of providing a variety of substituted anabaseines displaying a range of agonistic efficacies at alpha7 nicotinic receptors. Factors to be taken into consideration include disposition of the therapeutic target, whether CNS or peripheral within systemic circulation, or contained within an organ with unique access such as the lung; possible side effects of the alpha7 drug at sites other than the intended target as well as through the intended target; and the need for a highly selective agonist, in addition to the age, sex, and general health of the patient. For example, it may be advantageous to use an anabaseine derivative that does not cross the blood brain barrier when systemic and other peripheral inflammations are being treated and the alpha7 receptors on macrophages are being targeted. In treating pulmonary inflammation it may be preferable is to utilize an anabaseine that does not readily pass into the systemic circulation after being administered through an inhaler directly into the pulmonary space.

It is expected that the disclosed compounds may also exhibit pharmacokinetic as well as pharmacodynamic properties that are distinctly superior to previously synthesized and tested compounds and which would not have been predicted. Addition of a chemical group to improve compound potency, efficacy and selectivity may also make the compound less readily metabolized by protecting otherwise reactive sites on the molecule. For example, R² at position 4, R³ at position 5 and R⁴ at position 6 on the terahydropyridyl ring are methyl groups either singly substituted, for example, R² is a methyl group or each is substituted with methyl and are in (S)⁻ or (R)⁻ (alpha or beta) enantiomeric form. Thus, position of the substituents providing alpha7 selectivity may also improve the pharmacokinetic properties of the anabaseine.

It is believed that that alpha7 nicotinic receptor agonists may be useful in stimulating neoangiogenesis in wound healing and other conditions in which there is inadequate tissue perfusion. New tissue requires a robust blood supply in order to function efficiently and tissue lacking sufficient oxygenation may become necrotic. Development of new blood vessels is of prime importance in recovery of damaged heart tissue. The brain is the site of several types of insults, including stroke, vascular dementia and there is a decrease in number of microvessels in the aging brain (Uspenskaia, et al., 2004). In selected cases therefore, it may be beneficial to target cerebral microvessels in the basal lamina with the agents of the present invention in order to stimulate neoangiogenesis and increase blood flow and distribution in the brain.

Inhibition of angiogenesis would be desirable in certain medical conditions, such as in tumor cell proliferation and in some forms of retinal (macular) degeneration. Alpha7 nAChR antagonists could be useful in inhibiting angiogenesis, as new blood vessel growth is necessary for growth of solid tumors. An anabaseine alpha7 nAChR antagonist that is polar, and/or ionized and/or conjugated to another inactive molecule such as a complex carbohydrate or a polyethylene glycol that confers on the molecule pharmacokinetic advantages and limits its diffusion to the compartment of administration may be useful as angiogenesis inhibitor in treating certain conditions. Such an anabaseine type alpha7 nAChR antagonist could also be directly administered into the arterial blood perfusing the tumor to achieve even greater selectivity of action.

The invention is expected to be useful in a number of applications, particularly in treatment of diseases where it is advantageous to upregulate alpha7 nicotinic receptor activity. Loss of alpha7 receptors occurs in the progression of Alzheimer's disease and there is deficient expression of this receptor subtype in schizophrenia. It has been shown that chronic administration of alpha7 agonists like DMXBA can lead to an increased expression of functional alpha7 receptors on cell surfaces. Thus chronic administration of an alpha7-selective drug may have an even greater effect than before up-regulation in alpha7 number and responsiveness has occurred. In contrast to alpha7 selective lignands, alpha4beta2 receptor ligands generally cause a down-regulation of overall responsiveness of a cell while at the same time there may be an increase in alpha4beta2 receptor number. Thus chronic administration of alpha4beta2 agonists is more likely to cause tolerance. An up-regulation in responsiveness is expected with the compounds of the invention, either alone or in combination, in appropriate pharmaceutically acceptable forms.

The compounds of the invention are selective ligands (agonists or antagonists) of alpha7 nicotinic receptors, which have little or no activity with respect to other nACh receptor subtypes, particularly α4β2 receptors. Exemplary anabaseine compounds include compounds with substituents on one or more of the three ring systems present; i.e., pyridyl, tetrahydropyridyl and 3-arylidene. In a preferred embodiment, R² at position 4, R³ at position 5 and R⁴ at position 6 on the terahydropyridyl ring are methyl groups either singly substituted, for example, R² is a methyl group or each is substituted with methyl and are in (S)⁻ or (R)⁻ (alpha or beta) enantiomeric form.

It has been discovered that selection of a particular substituent to be placed in one of these rings can improve selectivity of binding for the alpha7 receptor and can also determine whether the occupied receptor will be activated or inhibited. For example, substitutions expected to provide these properties include methyl, propyl, ethyl, acetamido, acetoxy, alkoxy, alkyl, amino, aryl, benzofuran-2-ylmethylene, benzyl, carbamate, dimethylaminoalkoxy, modified glucuronidyl and 1H-indol-2-ylmethylene groups. Substitution at the alpha- or beta-oriented sites at positions 4, 5 and 6 of the tetrahydropyridyl ring form chiral products that display significantly improved alpha7 receptor selectivity in comparison with the corresponding racemic substituted compounds. In a preferred embodiment, R² at position 4, R³ at position 5 and R⁴ at position 6 on the terahydropyridyl ring are methyl groups either singly substituted, for example, R² is a methyl group or each is substituted with methyl and single substitutions are in (S)⁻ or (R)⁻ (alpha or beta) enantiomeric form.

In another preferred embodiment, R² at position 4, R³ at position 5 and R⁴ at position 6 on the terahydropyridyl ring are substituted with one or more of: methyl, propyl, ethyl groups.

Combinations of substituents on two or all three different ring portions of these anabaseine compounds are expected to provide even greater selectivity than when they are made individually on just one of the three ring structures.

Other objects of the invention may be apparent to one skilled in the art upon reading the following specification and claims.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 shows the chemical structures for the compounds GTS-2, GTS-3, GTS-5, GTS-7, GTS-13, GTS-20, GTS-26, GTS-27, GTS-28, GTS-35, GTS-38 and GTS-39.

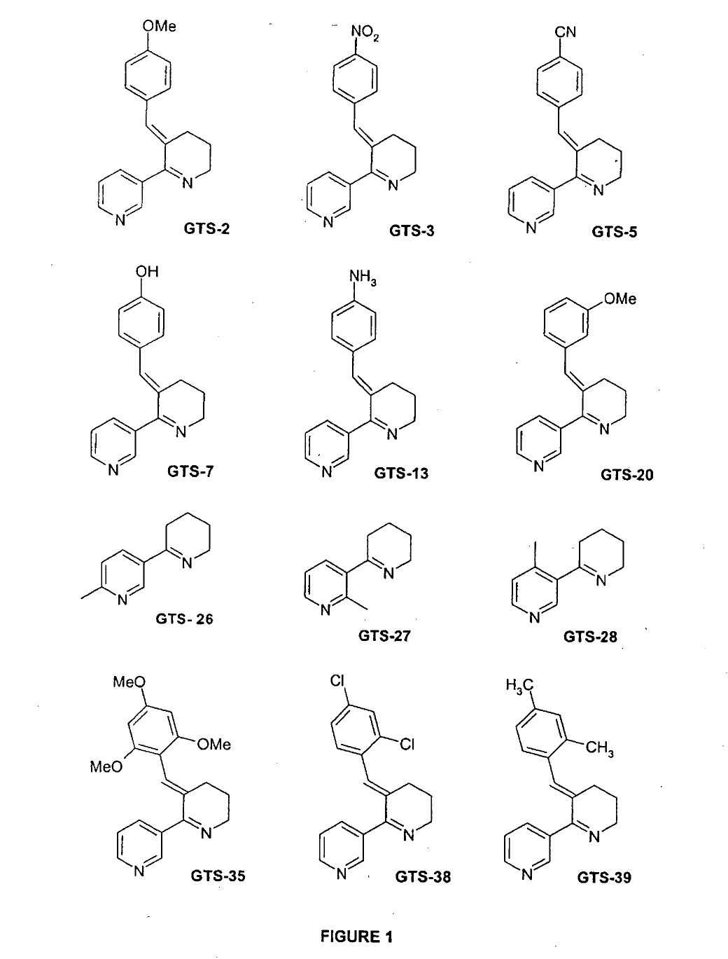

FIG. 2 shows the chemical structures for the compounds GTS-40, GTS-43, GTS-45, GTS-48, GTS-51, GTS-52, GTS-53, GTS-54 and GTS-55.

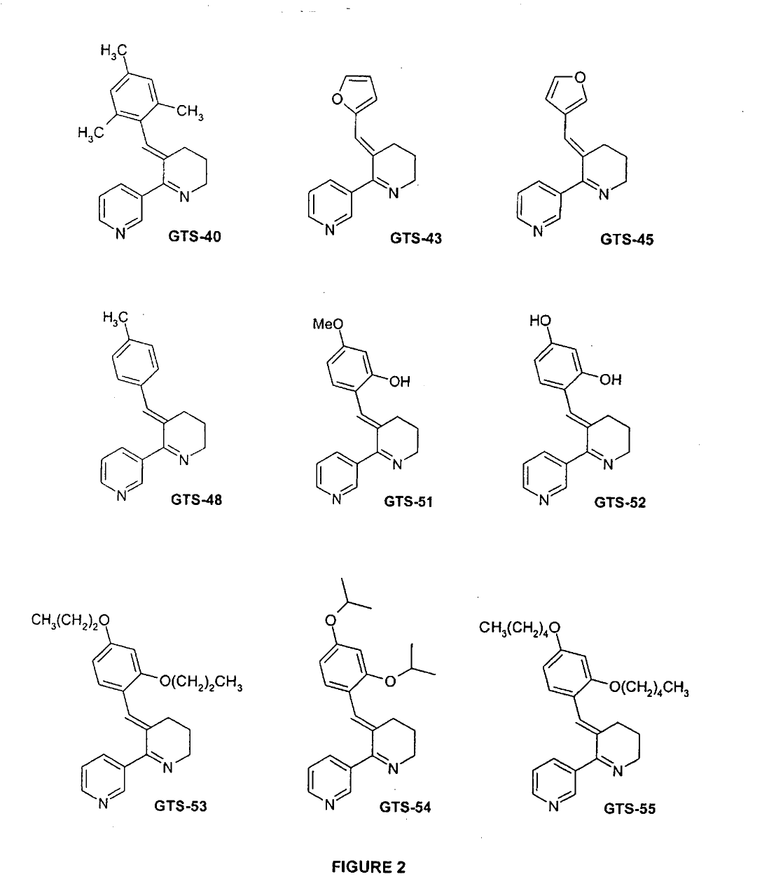

FIG. 3 shows the chemical structures for the compounds GTS-56, GTS-57, GTS-58, GTS-60, GTS-62, GTS-63, DMACA and GTS-21.

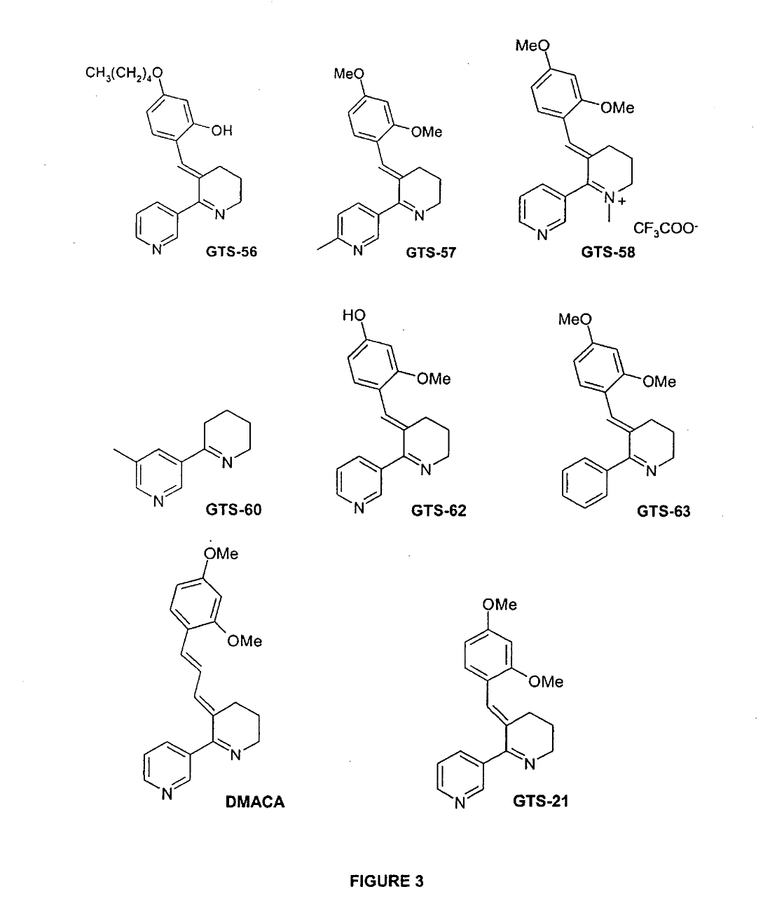

### DETAILED DESCRIPTION

Before the present invention is described, it is to be understood that this invention is not limited to particular methodologies (e.g., modes of administration) or specific compositions described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

It must be noted that as used herein and in the appended claims, the singular forms “a”, “and”, and “the” include plural referents unless the context clearly dictates otherwise. Thus, for example, reference to “anabaseine agonist” includes a plurality of such agonists, reference to “anabaseine antagonist” includes a plurality of such antagonists and reference to “the nicotine acetylcholine receptor” includes reference to one or more receptors and equivalents thereof known to those skilled in the art, and so forth.

As utilized in accordance with the present disclosure, the following terms, unless otherwise indicated, shall be understood to have the following meanings:

“Active ingredient” refers to an anabaseine agonist or an anabaseine antagonist of Formula I or II.

“Anabaseine agonist” refers to a compound of Formula I or II that binds substantially 15 specifically to a nicotinic cholinergic receptor (nAChR) and causes the receptor to be activated to provide a pharmacological effect, such as the induction of angiogenesis. Typically, activation of a nAChR causes its associated ion channel to open, resulting in calcium influx and membrane depolarization. This definition includes anabaseine partial agonists, which are compounds of Formula I and/or II, that when bound to a nAChR are less likely than a pure nicotinic agonist such as acetylcholine to cause activation, but activation does occur at least part of the time.

“Anabaseine antagonist” refers to a compound of Formula I and/or II that binds substantially specifically to a nicotinic cholinergic receptor (nAChR) but fails to cause its associated ion channel to open. However, this failure of the channel to open in turn results in a pharmacological effect, such as the reduction of angiogenesis. This definition includes partial anabaseine antagonists, which are compounds of Formula I and/or II, that when bound to a nAChR are less likely than a pure anabaseine antagonist to block activation, but blocked activation does occur at least part of the time.

“Compounds” as used herein refers to compounds of Formula I and/or II, and includes any specific compounds encompassed by generic formulae disclosed herein. The compounds may be identified either by their chemical structure and/or chemical name. When the chemical structure and chemical name conflict, the chemical structure is determinative of the identity of the compound. The compounds may contain one or more chiral centers and/or double bonds and therefore, may exist as stereoisomers, such as double-bond isomers (i.e., geometric isomers), enantiomers or diastereomers. Accordingly, when stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including cyclic imine form, cyclic iminium form, amino-keto form, ammonium-ketone form, and mixtures thereof. Accordingly, the chemical structures depicted herein encompass all possible tautomeric forms of the illustrated compounds. The compounds also include isotopically labeled compounds where one or more atoms have an atomic mass different from the atomic mass conventionally found in nature. Examples of isotopes that may be incorporated into the compounds include, but are not limited to, ²H, ³H, ¹³C, ¹⁴C, ¹⁵N, ¹⁷O and ¹⁸O. Compounds may exist in unsolvated forms as well as solvated forms, including hydrated forms and as N-oxides. In general, the hydrated, solvated and N-oxide forms are within the scope of the present disclosure. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Halo” means fluoro, chloro, bromo, or iodo.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, butyric acid, propionic acid, hexanoic acid, cyclopentanepropionic acid, glycolic acid, pyruvic acid, lactic acid, valeric acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like, made by conventional chemical means; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like, made by conventional chemical means.

“Pharmaceutically acceptable carrier” refers to a diluent, adjuvant, excipient or vehicle with which a compound is administered.

“Pharmaceutical composition” as used herein refers to at least one anabaseine agonist or anabaseine antagonist and a pharmaceutically acceptable carrier with which the anabaseine agonist or anabaseine antagonist is administered to a patient.

“Protecting group” refers to a grouping of atoms that when attached to a reactive functional group in a molecule masks, reduces or prevents reactivity of the functional group. Examples of protecting groups can be found in Green et al, “Protective Groups in Organic Chemistry”, (Wiley, 2nd ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), teri-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

The term “alkyl” refers to the radical of saturated aliphatic groups, including straight-chain alkyl groups, and branched-chain alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C1-C30 for straight chain, C3-C30 for branched chain), preferably 26 or fewer, and more preferably 20 or fewer, and still more preferably 4 or fewer.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate.

The term “alkyl” also includes unsaturated aliphatic groups analogous in length and possible substitution to the alkyls described above, but that contain at least one double or triple bond respectively. An “alkylaryl” moiety is an alkyl substituted with an aryl (e.g., phenylmethyl (benzyl)).

The terms “alkoxy,” “aminoalkyl” and “thioalkoxy” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aralkyl” means an aryl group that is attached to another group by a (C1-C6)alkylene group. Aralkyl groups may be optionally substituted, either on the aryl portion of the aralkyl group or on the alkylene portion of the aralkyl group, with one or more substituents.

The term “aryl” as used herein, refers to the radical of aryl groups, including 5- and 6-membered single-ring aromatic groups that may include from zero to four heteroatoms (heteroaryl), for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like.

Those aryl groups having heteroatoms in the ring structure may also be referred to as “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, halogenated alkyl (including trifluoromethyl, difluoromethyl and fluroromethyl), halogenated alkoxy (including trifluoromethoxy, difluoromethoxy and fluroromethoxy), cyano, azido, heterocyclyl, alkylaryl, arylalkyl or an aromatic or heteroaromatic moiety. Aryl groups can also be fused or bridged with alicyclic or heterocyclic rings which are not aromatic so as to form a polycycle (e.g., tetralin).

The term “cyclyl” refers to a hydrocarbon 3-8 membered monocyclic or 7-14 membered bicyclic ring system having at least one non-aromatic ring, wherein the non-aromatic ring has some degree of unsaturation. Cyclyl groups may be optionally substituted with one or more substituents. In one embodiment, 0, 1, 2, 3, or 4 atoms of each ring of a cyclyl group may be substituted by a substituent. The term “cycloalkyl” refers to a hydrocarbon 3-8 membered monocyclic or 7-14 membered bicyclic ring system having at least one saturated ring. Cycloalkyl groups may be optionally substituted with one or more substituents. In one embodiment, 0, 1, 2, 3, or 4 atoms of each ring of a cycloalkyl group may be substituted by a substituent. Cycloalkyls can be further substituted, e.g., with the substituents described above. Preferred cyclyls and cycloalkyls have from 3-10 carbon atoms in their ring structure, and more preferably have 3, 4, 5, 6 or 7 carbons in the ring structure. Those cyclic groups having heteroatoms in the ring structure may also be referred to as “heterocyclyl,” “heterocycloalkyl” or “heteroaralkyl.” The aromatic ring can be substituted at one or more ring positions with such substituents as described above.

The terms “cyclyl” or “cycloalkyl” refer to the radical of two or more cyclic rings (e.g., cycloalkyls, cycloalkenyls, cycloalkynyls, aryls, heteroaryls, and/or heterocyclyls). In some cases, two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, halogenated alkyl (including trifluoromethyl, difluoromethyl and fluroromethyl), halogenated alkoxy (including trifluoromethoxy, difluoromethoxy and fluroromethoxy), cyano, azido, heterocyclyl, alkyl, alkylaryl, or an aromatic or heteroaromatic moiety.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “methyl” refers to a CH₃ group. In a preferred embodiment, R² at position 4, R³ at position 5 and R⁴ at position 6 on the terahydropyridyl ring are methyl groups either singly substituted, for example, R² is a methyl group or each is substituted with methyl and are in (S)⁻ or (R)⁻ (alpha or beta) enantiomeric form.

The term “mercapto” refers to a SH group.

The term “sulfhydryl” or “thiol” means —SH.

The compounds of the invention encompass various isomeric forms. Such isomers include, e.g., stereoisomers, e.g., chiral compounds, e.g., diastereomers and enantiomers.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

Furthermore the indication of configuration across a carbon-carbon double bond can be “Z” referring to what is often referred to as a “cis” (same side) conformation whereas “E” refers to what is often referred to as a “trans” (opposite side) conformation. Regardless, both configurations, cis/trans and/or Z/E are contemplated for the compounds for use in the present invention.

With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

Natural amino acids represented by the compounds utilized in the present invention are in the “L” configuration, unless otherwise designated. Unnatural or synthetic amino acids represented by the compounds utilized in the present invention may be in either the “D” or “L” configurations. Similarly glycosidic bonds may be in either alpha- or beta-configuration

Another aspect is a radiolabeled compound of any of the formulae delineated herein. Such compounds have one or more radioactive atoms (e.g., ³H, ²H, ¹⁴C, ¹³C, ³⁵S, ³²P, 1²⁵I, ¹³¹I) introduced into the compound. Such compounds are useful for drug metabolism studies and diagnostics, as well as therapeutic applications.

The term “prodrug” includes compounds with moieties, which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19; Silverman (2004) The Organic Chemistry of Drug Design and Drug Action, Second Ed., Elsevier Press, Chapter 8, pp. 497-549). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halogen, or methoxy substituents) aryl and aryl-lower alkyl esters, amides, lower-alkyl amides, di-lower alkyl amides, and hydroxy amides. Preferred prodrug moieties are propionoic and succinic acid esters, acyl esters and substituted carbamates. Prodrugs which are converted to active forms through other mechanisms in vivo are also included.

Alpha7 nAChRs have also been found on non-neuronal cells within the nervous system (for example, astrocytes and microglia) and outside the nervous system; e.g., macrophages, bronchial epithelium and vascular endothelium. Alpha7 receptors on peripheral macrophages, when stimulated by appropriate agonists, inhibit the secretion of cytokines, including tumor necrosis factor alpha (TNF), which cause inflammation. Similarly, stimulation of alpha7 nAChRs in vascular endothelium enhances the formation of new blood vessels (angiogenesis), an important process in wound healing. On the other hand, proliferation of certain small cell lung cancers expressing primarily alpha7 nAChRs can be stimulated by nicotinic agonists and possibly inhibited with certain nicotinic antagonists. Thus, besides being implicated as useful therapeutic targets for treating nervous system disorders such as Alzheimer's disease and schizophrenia, alpha7 nAChRs on non-neuronal cells may also be therapeutic targets for treating other disease states involving inflammation, trauma, deficient or excessive angiogenesis, and abnormal proliferation (cancer).

“Substituted” refers to a group in which one or more hydrogen atoms are each 5 independently replaced with the same or different substituent(s). Typical substituents include, but are not limited to, alkyl and N,N-dialkylamino.

“Treat”, “treating” and “treatment” all refer to obtaining a desired pharmacologic and/or physiologic effect, e.g., stimulation of angiogenesis and/or vasculogenesis or the inhibition of angiogenesis. The effect may be prophylactic in terms of completely or partially preventing a disease or symptom thereof and/or may be therapeutic in terms of a partial or complete cure for a disease and/or adverse effect attributable to the disease. For embodiments of the invention involving stimulation of angiogenesis, treatment” as used herein covers any treatment of a disease in a mammal, particularly a human, and includes: (a) preventing a disease or condition (e.g., preventing the loss of a skin graft or a re-attached limb due to inadequate vascularization) from occurring in a subject who may be predisposed to the disease but has not yet been diagnosed as having it; (b) inhibiting the disease, e.g., arresting its development; or (c) relieving the disease (e.g., enhancing the development of a “bio-bypass” around an obstructed vessel to improve blood flow to an organ or enhancing wound healing in an ischemic limb). In the context of the present invention, stimulation of angiogenesis and/or vasculogenesis is employed for subject having a disease or condition amenable to treatment by increasing vascularity and increasing blood flow. For embodiments of the invention involving inhibition of angiogenesis, “treatment” as used herein covers any treatment of a disease in a mammal, particularly a human, and includes: (a) preventing a disease or condition (e.g., preventing proliferative retinopathy) from occurring in a subject who may be predisposed to the disease but has not yet been diagnosed as having it; (b) inhibiting the disease (e.g., inhibiting further growth of a tumor); or relieving the disease. In the context of the present invention, reduction of angiogenesis and/or vasculogenesis is employed for subject having a disease or condition amenable to treatment by reducing angiogenesis.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Therapeutically effective amount” means the amount of a compound that, when administered to a patient for controlling angiogenesis, is sufficient to effect such control.

The “therapeutically effective amount” will vary depending on the compound, the severity of the condition causing the need to control angiogenesis and the age, weight, etc., of the patient to be treated. The term “anti-angiogenic activity” as used herein, refers to the inhibition and/or moderation of angiogenesis.

The term “angiogenesis-associated disease” is used herein, for purposes of the specification and claims, to mean certain pathological processes in humans where angiogenesis is abnormally prolonged. For example, angiogenesis-associated diseases include diabetic retinopathy, chronic inflammatory diseases, rheumatoid arthritis, dermatitis, psoriasis, stomach ulcers, tumors and the like.

Reference will now be made in detail to certain preferred methods of treatment, compounds and methods of administering these compounds. The invention is not limited to those preferred compounds and methods but rather is defined by the claim(s) issuing herefrom.

Compounds

The present invention provides methods for controlling angiogenesis in a compromised mammalian host by administering a therapeutically effective amount of an anabaseine compound of Formula I and/or II. Controlling angiogenesis includes both increasing and decreasing formation of vasculature, depending upon whether enhancement or diminishment provides a positive outcome in the particular disease or disorder being treated.

Although nicotinic cholinergic receptors (nAChRs) in the brain have long been recognized as being important in mediating the euphoric effects of nicotine, they attracted additional interest when significant nAChR deficits, later identified as primarily of the α₄β₂ receptor subtype, were discovered in postmortem brain samples from patients with Alzheimer's disease (M. N. Sabbagh et al, J. Neural Transm., 1998, 105:709-717). Mammalian nAChRs are pentameric ligand-gated ion channels that, upon activation, allow the movement of cations including calcium across the cell membrane. Besides causing membrane depolarization, nAChR (especially the α7 type)-mediated influx of calcium into the cell stimulates several signal transduction pathways (T. Kihara et al., J. Biol Chem., 2001, 276:13541-13546; F. A. Dajas-Bailador et al, J. Neurochem., 2002, 80:520-530). A variety of the nAChR subtypes are now known to be present in the mammalian brain. The two most abundant brain nAChRs are the α₄β₂ and homomeric α7 subtypes. The former contributes >90% of the high-affinity binding sites for nicotine in the rat brain (G. M. Flores et al., Mol Pharmacol, 1992, 41:31-37). The low-nicotine-affinity α7 nAChR is recognized by its nanomolar affinity for α-bungarotoxin (BTX) (M. J. Marks and A. C. Collins, Mol Pharmacol, 1982, 22:554-564).

Anabaseine is an animal toxin that, like nicotine, stimulates all nAChRs (W. R. Kem et al., J. Pharmacol. Exp. Ther., 1997, 283:979-992). DMXBA, 3-[(2,4-dimethoxy)benzylidene]-anabaseine hydrochloride, also known as GTS-21, a synthetic benzylidene derivative of anabaseine, selectively stimulates α7 subunit-containing nAChRs (C. M. de Fiebre et al., Mol. Pharmacol, 1995, 47:164-171; E. M. Meyer et al, J. Pharmacol. Exp. Ther., 1998, 287:918-925). Its efficacy (maximum effect) for activating the rat α7 receptor is approximately half that observed for acetylcholine and anabaseine, which are full agonists. DMXBA efficacy at the human α7 is only approximately half of the acetylcholine maximal response (Meyer et al, 1998). DMXBA displays neuroprotective properties (E. J. Martin et al, Drug Dev. Res., 1994, 31:134-141; T. Kihara et al, Ann. Neurol., 1997, 42:159-163; S. Shimohama et al, Brain Res., 1998, 779:359-363). DMXBA did not display significant human toxicity in a phase I clinical trial directed toward memory enhancement (H. Kitagawa et al, Neuropsychopharmacol 2003, 28:542-551).

Anabaseine's non-aromatic tetrahydropyridine ring imine double bond is conjugated with π-electrons of the 3-pyridyl ring. The imine nitrogen is a much weaker base than the pyrrolidinyl nitrogen of nicotine (Yamamoto, et al, Agr. Biol Chem., 1962, 26:709). Considerable evidence (Barlow and Hamilton, Brit. J. Pharmacol., 1962, 18:543) exists that the non-aromatic ring nitrogen of nicotine must be protonated (cationic) in order to avidly bind to the skeletal muscle nicotinic receptor and activate the opening of its channel. At physiological pH, anabaseine also exists in a hydrolyzed ammonium-ketone form as well as the cyclic imine (unionized) and cyclic iminium (monocationic) forms. Kem (“Nemertine Toxins,” in Animal Toxins. Tools in Cell Biology, eds., H. Rochat and M.-F. Martin-Eauclaire, Chapman and Hail, pp. 57-73) has determined that anabaseine acts as a central nicotinic receptor agonist primarily through its cyclic iminium form.

Throughout this specification, whenever reference is made to a compound of Formula I and/or II wherein there is a double bond between the 1-and 2-position in the B ring, it is to be understood that this also refers to an open (noncyclic) ammonium ketone form corresponding to the cyclic imine or cyclic iminium form of the Formula I and/or II.

Suitable anabaseine agonists and anabaseine antagonists for use in the methods of the invention include, but are not limited to, compounds of the formula:

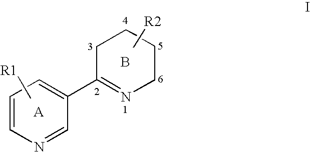

or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, propyl, ethyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or

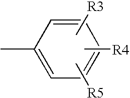

wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is

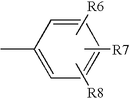

wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; or a pharmaceutically acceptable salt, solvate, clathrate, stereoisomer, enantiomer, prodrug or combinations thereof, wherein the compound of Formula I and/or II functions as either an anabaseine agonist or an anabaseine antagonist. In the embodiments of the invention involving methods for induction of angiogenesis in a mammal, the compounds of Formula I and/or II that function as anabaseine agonists are administered. In the embodiments of the invention involving methods of reducing angiogenesis in a mammal, the compounds of Formula I and/or II that function as anabaseine antagonists are administered.

In a preferred embodiment, R² at position 4, R³ at position 5 and R⁴ at position 6 on the terahydropyridyl ring are methyl, propyl, ethyl groups either singly substituted, for example, R² is a methyl group or each is substituted with methyl and are in (S)⁻ or (R)⁻ (alpha or beta) enantiomeric form.

Preferred compounds for use in the methods of the invention include:

GTS-2, 3-(4-Methoxybenzylidene)anabaseine

GTS-3, 3-(4-Nitrobenzylidene)anabaseine

GTS-5, 3-(4-Cyanobenzylidene)anabaseine

GTS-7, 3-(4-Hydroxybenzylidene)anabaseine

GTS-8, 3-(4-Chlorobenzylidene)anabaseine

GTS-13,3-(4-Aminobenzylidene)anabaseine

GTS-15, 3-(4-Dimethylaminopropoxy-benzylidene)anabaseine

GTS-16, 3-(2-Methoxybenzylidene)anabaseine

GTS-20, 3-(3-Methoxybenzylidene)anabaseine

GTS-21, DMXBA, 3-(2,4-Dimethoxybenzylidene)anabaseine.

GTS-23, 3-(3-Methoxy-4-hydroxybenzylidene)anabaseine

GTS-26, 6′-Methylanabaseine;

GTS-27, 2′-Methylanabaseine;

GTS-28, 4′-Methylanabaseine;

GTS-35, 3-(2,4,6-Trimethoxybenzylidene)anabaseine

GTS-38, 3-(2,4-Dichlorobenzylidene)anabaseine.

GTS-39, 3-(2,4-Dimethylbenzylidene)anabaseine

GTS-40, 3-(2,46,-Trimethylbenzylidene)anabaseine

GTS-43, 3-(2-Furylidene)anabaseine

GTS-44, 3-(2-Furylpropenylidene)anabaseine

GTS-45, 3(3-Furylidene)anabaseine

GTS-48, 3-(4-Methylbenzylidene)anabaseine

GTS-51 , 3-(2-Hydroxy-4-methoxybenzylidene)anabaseine

GTS-52, 3-(2,4-Dihydroxybenzylidene)anabaseine

GTS-53, 3-(2,4-Dipropoxybenzylidene)anabaseine

GTS-54, 3-(2,4-Diisopropoxybenzylidene)anabaseine

GTS-55, 3-(2,4-Dipentoxybenzylidene)anabaseine

GTS-56, 3-(2-Hydroxy-4-pentoxybenzylidene)anabaseine

GTS-57, 6′-Methyl-3-(2,4-dimethoxybenzylidene)anabaseine

GTS-58, 1-Methyl-3-(2,4-djmethoxybenzylidene)anabaseine trifluoroacetate;

GTS-60, 5′-Methylanabaseine;

GTS-62, 3-(2-Methoxy-4-hydroxybenzylidene)anabaseine

GTS-63, 2-Phenyl-3-(2,4-dimethoxybenzylidene)-4,5,6-trihydropyridine; and DMACA, 3-(4-Dimethylaminocinnamylidene)anabaseine.

GTS-83

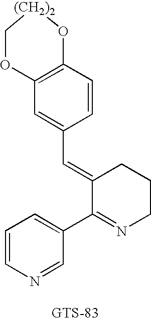

The invention also encompasses the rational development of new compounds which exhibit significantly enhanced alpha7 nAChR selectivity, relative to these basic structures. Because of their greatly enhanced selectivity toward alpha7 receptors, these new structures and compounds containing the important elements of these structures will provide a panel of useful therapeutic agents that can be targeted not only to specific diseases, but also to particular areas of the body; for example, to the CNS for neurodegenerative conditions or to peripheral areas in cases of systemic inflammation.

In a preferred embodiment, anabaseine agonists and anabaseine antagonists for use in the methods of the invention include, but are not limited to, compounds of the formula:

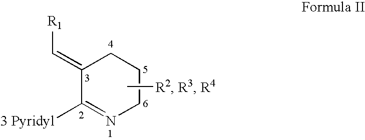

or a pharmaceutically acceptable salt thereof; wherein R¹ is hydrogen, methyl, propyl, ethyl, acetoxy, acetamido, amino, dimethylcarbamate, dimethylaminopropoxy, hydroxyl, methoxy, isopropoxy, trifluromethoxy or thiomethoxy or C₁-C₄ alkyl; and R² is hydrogen, methyl, propyl, ethyl, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or,

wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; wherein the compound of Formula I functions as either an anabaseine agonist or an anabaseine antagonist.

In a preferred embodiment, R² at position 4, R³ at position 5 and R⁴ at position 6 on the terahydropyridyl ring are methyl, propyl, ethyl groups either singly substituted, for example, R² is a methyl group or each is substituted with methyl and are in (S)⁻ or (R)⁻ (alpha or beta) enantiomeric form.

The present invention shows that selection of appropriate substituents on the tetrahydropyridyl a ring portions of anabaseine compounds determines alpha7 selectivity, either when done separately or in combinations. Exemplarysubstitutions are: R² at position 4, R³ at position 5 and R⁴ at position 6 on the terahydropyridyl ring are methyl groups either singly substituted, for example, R² is a methyl group or each is substituted with methyl and are in (S)⁻ or (R)⁻ (alpha or beta) enantiomeric form.

Certain substituents also determine alpha7 receptor efficacy: Some substituents increase efficacy over benzylidene-anabaseines such as DMXBA while other reduce efficacy to essentially zero, thereby creating a new group of alpha7 nAChR antagonists.

Possible applications of these new alpha7 agonists and antagonists based on the anabaseine structure include therapeutic treatments for neurodegenerative diseases and nicotinic receptor involved addictions as well as potential for development as antiproliferation drugs. In particular, it is shown that altering anabaseine compound polarity and ionization can permit drug application and localization to the peripheral (blood and interstitial fluid) compartments without significant entry into the central nervous system.

In addition to CNS applications, this invention is expected to provide therapeutic agents that selectively stimulate peripheral alpha7 receptors expressed on non-neuronal cells such as macrophages, vascular endothelium and bronchial epithelium, which are peripheral cells known to express functional alpha7 nAChRs. When macrophage alpha7 receptors are stimulated, the secretion of inflammatory cytokines such as TNF is inhibited. These cytokines are known to exacerbate an immune response when overproduced and not efficiently removed from the system. Stimulation of vascular endothelial cells, for example, is known to enhance angiogenesis.

It is believed that that alpha7 nicotinic receptor agonists may be useful in stimulating neoangiogenesis in wound healing and other conditions in which there is inadequate tissue perfusion. New tissue requires a robust blood supply in order to function efficiently and tissue lacking sufficient oxygenation may become necrotic. Development of new blood vessels is of prime importance in recovery of damaged heart tissue. The brain is the site of several types of insults, including stroke, vascular dementia and there is a decrease in number of microvessels in the aging brain (Uspenskaia, et al., 2004). In selected cases therefore, it may be beneficial to target cerebral microvessels in the basal lamina with the agents of the present invention in order to stimulate neoangiogenesis and increase blood flow and distribution in the brain.

Inhibition of angiogenesis would be desirable in certain medical conditions, such as in tumor cell proliferation and in some forms of retinal (macular) degeneration. Alpha7 nAChR antagonists could be useful in inhibiting angiogenesis, as new blood vessel growth is necessary for growth of solid tumors. An anabaseine alpha7 nAChR antagonist that is polar, and/or ionized and/or conjugated to another inactive molecule such as a complex carbohydrate or a polyethylene glycol that confers on the molecule pharmacokinetic advantages and limits its diffusion to the compartment of administration may be useful as angiogenesis inhibitor in treating certain conditions. Such an anabaseine type alpha7 nAChR antagonist could also be directly administered into the arterial blood perfusing the tumor to achieve even greater selectivity of action.

The invention is expected to be useful in a number of applications, particularly in treatment of diseases where it is advantageous to upregulate alpha7 nicotinic receptor activity. Loss of alpha7 receptors occurs in the progression of Alzheimer's disease and there is deficient expression of this receptor subtype in schizophrenia. It has been shown that chronic administration of alpha7 agonists like DMXBA can lead to an increased expression of functional alpha7 receptors on cell surfaces. Thus chronic administration of an alpha7-selective drug may have an even greater effect than before up-regulation in alpha7 number and responsiveness has occurred. In contrast to alpha7 selective lignands, alpha4beta2 receptor ligands generally cause a down-regulation of overall responsiveness of a cell while at the same time there may be an increase in alpha4beta2 receptor number. Thus chronic administration of alpha4beta2 agonists is more likely to cause tolerance. An up-regulation in responsiveness is expected with the compounds of the invention, either alone or in combination, in appropriate pharmaceutically acceptable forms.

The compounds of the invention are selective ligands (agonists or antagonists) of alpha7 nicotinic receptors, which have little or no activity with respect to other nACh receptor subtypes, particularly α4β2 receptors. Exemplary anabaseine compounds include compounds with substituents on one or more of the three ring systems present; i.e., pyridyl, tetrahydropyridyl and 3-. It has been discovered that selection of a particular substituent to be placed in one of these rings can improve selectivity of binding for the alpha7 receptor and can also determine whether the occupied receptor will be activated or inhibited. For example, substitutions expected to provide these properties include acetamido, acetoxy, alkoxy, alkyl, amino, aryl, benzofuran-2-ylmethylene, benzyl, carbamate, dimethylaminoalkoxy, modified glucuronidyl and 1H-indol-2-ylmethylene groups. Substitution at the alpha- or beta-oriented sites at positions 4, 5 and 6 of the tetrahydropyridyl ring form chiral products that display significantly improved alpha7 receptor selectivity in comparison with the corresponding racemic substituted compounds. Combinations of substituents on two or all three different ring portions of these anabaseine compounds are expected to provide even greater selectivity than when they are made individually on just one of the three ring structures.

In a preferred embodiment, R² at position 4, R³ at position 5 and R⁴ at position 6 on the terahydropyridyl ring are methyl groups either singly substituted, for example, R² is a methyl group or each is substituted with methyl and single substitutions are in (S)⁻ or (R)⁻ (alpha or beta) enantiomeric form.

In another preferred embodiment, R² at position 4, R³ at position 5 and R⁴ at position 6 on the terahydropyridyl ring are substituted with one or more of: methyl, propyl, ethyl groups.

Other objects of the invention may be apparent to one skilled in the art upon reading the following specification and claims.

Chemical Synthesis of Compounds of Formula I and/or II

The compounds of Formula 1 may be prepared by chemical synthesis according to the methods disclosed in U.S. Pat. No. 5,616,785, issued May 14, 1996; U.S. Pat. No. 5,741,802, issued Apr. 21, 1998; U.S. Pat. No. 5,977,144, issued Nov. 2, 1999; and U.S. Pat. No. 6,630,491, issued Oct. 7, 2003.

Briefly, the compounds of Formula I and/or II wherein R² is other than ═CH—X or ═CHCH═CH-Z may be prepared by reacting the appropriate protected 2-piperidone with an appropriate pyridyl lithium or phenyl lithium. Pyridyl lithium may be prepared from the corresponding bromopyridine (H. Gilman, et al, J. Org. Chem., 1951, 16:1485). Typically, the pyridyl lithium, which is freshly prepared, is used in the condensation in an inert solvent, e.g., dry ether. The reaction is usually complete within a few hours. The reaction mixture is then acidified and the product is isolated by solvent extraction and purified by, for example, recrystallization.

The compounds of Formula I and/or II wherein R² is ═CH—X may be prepared from anabaseine. In general, a solution of anabaseine (or its dihydrochloride) in acetic acid is treated with about two molar equivalents of an aldehyde (X—CHO), and the resulting mixture is heated to approximately 60° C. for about 24 hours. The compounds of Formula I and/or II can be isolated and purified by standard techniques such as chromatography and recrystallization.

Although the above acidic reaction conditions are generally satisfactory, basic reaction conditions or buffered conditions are required in the case of the reacting aldehydes bearing an electron-withdrawing group such as nitro. Thus, a basic agent can also be used in the mixed aldol-type condensation.

The compounds of Formula I and/or II wherein X is substituted or unsubstituted phenyl can adopt two conformations about the double bond at the 3-position. Although the E isomer is preferred, a Z isomer also exists. Both E and Z isomers are considered to be within the purview of the present invention.

The compounds of Formula I and/or II wherein R² is ═CHCH═CH-Z may be prepared as disclosed in U.S. Pat. No. 5,911,144, issued Nov. 2, 1999, which is herein incorporated by reference.

The compounds of Formula I and/or II in their free base form will form acid addition salts, and these acid addition salts are non-toxic and pharmaceutically acceptable for therapeutic use. The acid addition salts are prepared by standard methods, for example by combining a solution of anabaseine in a suitable solvent (e.g., water, ethyl acetate, acetone, methanol, ethanol or butanol) with a solution containing a stoichiometric equivalent of the appropriate acid. If the salt precipitates, it is recovered by filtration. Alternatively, it can be recovered by evaporation of the solvent or, in the case of aqueous solutions, by lyophilization. Of particular value are the sulfate, hydrochloride, hydrobromide, nitrate, phosphate, citrate, tartrate, pamoate, perchlorate, sulfosalicylate, benzene sulfonate, 4-toluene sulfonate and 2-naphthalene sulfonate salts. These acid addition salts are considered to be within the scope and purview of this invention.

Angiogenesis and Anti-Angiogenesis

The angiogenic and ant-angiogenic activities of the compounds of the invention can be measured by methods well known in the art. For example Heeschen C et al., (2002) J. Clin. Invest. 110(4):527-536, incorporated herein by reference.

Angiogenesis can also be induced on the chick chorioallantoic membrane (CAM) after normal embryonic angiogenesis has resulted in the formation of mature blood vessels. Angiogenesis has been shown to be induced in response to specific cytokines or tumor fragments as described by Leibovich et al., Nature, 329:630 (1987) and Ausprunk et al., Am. J Pathol., 79:597 (1975). CAMs were prepared from chick embryos for subsequent induction of angiogenesis and inhibition thereof. Ten day old chick embryos are incubated at 99.5° Fahrenheit with 60% humidity. A small hole is made through the shell at the end of the egg directly over the air sac with the use of a small crafts drill (Dremel, Division of Emerson Electric Co. Racine Wis.). A second hole is drilled on the broad side of the egg in a region devoid of embryonic blood vessels determined previously by candling the egg. Negative pressure is applied to the original hole, resulting in the CAM (chorioallantoic membrane) pulling away from the shell membrane and creating a false air sac over the CAM. A 1.0 centimeter (cm)×1.0 cm square window is cut through the shell over the dropped CAM with the use of a small model grinding wheel (Dremel). The small window allows direct access to the underlying CAM.

The resultant CAM preparation is then either used at 6 days of embryogenesis, a stage marked by active neovascularization, without additional treatment to the CAM reflecting the model used for evaluating effects on embryonic neovascularization or used at 10 days of embryogenesis where angiogenesis has subsided. The latter preparation is thus used in this invention for inducing renewed angiogenesis in response to treatment by the compounds of the invention.

Histology of the CAM: To analyze the microscopic structure of the chick embryo CAMs, about 6 micron thick sections are cut from the frozen blocks on a cryostat microtome for immunofluorescence analysis.

CAM Angiogenesis Assay: Angiogenesis is induced by placing a 5 millimeter (mm)×5 mm Whatman filter disk (Whatman Filter paper No. 1.) saturated with Hanks Balanced Salt Solution (HBSS) or HBSS containing 150 nanograms/milliliter (ng/ml) of known angiogenic factors e.g. recombinant basic fibroblast growth factor (βFGF) (Genzyme, Cambridge, Mass.) on the CAM of a 10-day chick embryo in a region devoid of blood vessels and the windows were latter sealed with tape. Compounds of the invention (e.g. Formula I and II) are run in parallel. Angiogenesis is monitored by photomicroscopy after 72 hours. CAMs are snap frozen, and 6 μm cryostat sections and fixed with acetone and stained by immunofluorescence.

Embryonic Angiogenesis: The CAM preparation for evaluating the effect of angiogenesis inhibitors on the natural formation of embryonic neovasculature is the 6 day embryonic chick embryo as previously described. At this stage in development, the blood vessels are undergoing de novo growth and thus provides a useful system for determining any compounds participating in embryonic angiogenesis.

Angiogenesis Induced by Tumors: To investigate the role of the compounds in tumor-induced angiogenesis, αᵥβ₃-negative human M21-L melanoma fragments or any other cells isolated from tumors are used in the CAM assay. These tumors can be grown and isolated from the CAM of a 17-day chick embryo as described by Brooks et al., J. Cell Biol., 122:1351 (1993). These fragments induced extensive neovascularization in the presence of buffer alone.

Angiogenesis is induced in the CAM assay system by direct apposition of a tumor fragment on the CAM. Preparation of the chick embryo CAM is essentially the same as the procedure described above. Instead of a filter paper disks a 50 milligram (mg) to 55 mg in weight fragment of either human melanoma tumor M21L or human lung carcinoma tumor UCLAP-3, both of which are αᵥβ₃ negative tumors, is placed on the CAM in an area originally devoid of blood vessels.

The M21L human melanoma cell line or the UCLAP-3 human lung carcinoma cell line, can be used to grow the solid human tumors on the CAMs of chick embryos. A single cell suspension of 5×10⁶ M21L or UCLAP-3 cells is first applied to the CAMs in a total volume of 30 microliters (μl) of sterile HBSS. The windows are sealed with tape and the embryos were incubated for 7 days to allow growth of human tumor lesions. At the end of 7 days, now a 17-day embryo, the tumors are resected from the CAMs and trimmed free of surrounding CAM tissue. The tumors are sliced into 50 mg to 55 mg tumor fragments. The tumor fragments were placed on a new set of 10 day chick embryo CAMs in an area devoid of blood vessels.

Inhibition of Angiogenesis as Measured in the CAM Assay: To determine the anti-angiogenic effects of the compounds of the invention, filter disks saturated with the compounds disclosed herein, βFGF or TNFα are placed on CAMs.

At 72 hours, CAMs are harvested and placed in a 35 mm petri dish and washed once with 1 ml of phosphate buffered saline. The bottom side of the filter paper and CAM tissue is then analyzed under an Olympus stereo microscope, with two observers in a double-blind fashion. Angiogenesis inhibition is considered significant when CAMs exhibited >50% reduction in blood vessel infiltration of the CAM directly under the disk.

Other Animal Models: The in vivo effects of the compounds of the invention can be assayed through various animal models known to those of ordinary skill in the art. Generally such assays involve the injection of a carcinoma cell line, of mouse or preferably, human, origin, into a cohort of mice. Following the passage of several days, as determined by the proliferative rate of the cell line, parameters such as tumor size, degree of metastasis and cellular infiltration into a region in the vicinity of the tumor site are evaluated. In most experiments, two groups of experimental mice are studied: a first, control, group which receives only the cell line but no agent of Formula I or II, and a second, test, group which receives at least one agent of Formula I or II such as GST-21. This second group is divided into several subgroups each of which receives a different dose of the agent, preferably between 10 μg and 100 μg per day for 10-20 days. On these days, the control group will be administered control doses containing only vehicle with no active agent. The evaluation of tumor growth and organ specific metastasis will vary according to the tumor type studied.

In yet other assay systems aimed at determining the effect of the agents on metastatic spread or development, the agents are administered at the time of inoculation of the malignant cells or short thereafter. Similarly, the agents may be administered after the inoculation, but before the full development of the tumor mass. In these ways, the effects of agents on different stages of malignant growth and metastasis can be tested.

The following are examples of different in vivo model systems for studied a variety of epithelial cancers.

Lung carcinoma: One such example for lung carcinoma involves the subcutaneous flank injection of the M109 mouse lung tumor cell line into syngeneic mice. On each of days 4 through 8 after injection of the cell line, the mice receive single bolus daily doses of the compounds of the invention by tail vein injection. The compounds of the invention are prepared and administered in a carrier solution which is physiologically compatible with both the recipient environment and the stability of the compound. A preferable carrier solution is D-PBS with a carrier protein such as albumin. Mice are sacrificed at day 4 after cell line injection and at two day intervals after the administration of inhibitory compounds. The tumors are excised and weighed. Measurement data can be standardized relative to initial body weight of the recipient mouse.

Alternatively, if the transplantable tumor line is able to grow to the extent that it causes a reproducible and significant effect on total mouse body weight, than the recipients need not be sacrificed. In this readout system, starting at day 0 (i.e., prior to the introduction of the cell line), the mice are weighed daily to determine the tumor burden and to evaluate the effect of the injected compound(s) on tumor burden. Tumor mass can be calculated by the difference in mouse body mass during the experiment and at day 0. Measurement of control mice which receive only carrier solution with carrier protein will. At be used to standardize for any unrelated weight gain.

Colon carcinoma: The effect of test agents on experimentally induced human colorectal tumors in mice can be deduced by transplanting into nude mice human colon tumor cell lines such as COLO 205, C-1H, 26M3.1, CT-26, LS174T, and HT29, in a manner similar to that described above. In these models, pericecal tumor growth, angiogenesis, ascites and metastasis to the liver are suitable readouts to ascertain if the test compounds are active.

Melanoma and metastatic melanoma: Melanoma cell lines (e.g., B16 and SKMEL) are administered either intraperitoneally or intravenously or directly into the footpad. Primary tumor growth, survival time, resistance to tumor challenge, cellular infiltrates characteristic of melanoma tumors, and extent of tumor angiogenesis are all parameters of interest which can be evaluated. In certain models of melanoma, metastasis to the lung can be readily observed following surgical removal of the primary tumor.

Ovarian cancer: Human ovarian carcinoma cell lines such as JAM are administered subcutaneously to severe combined immunodeficiency (SCID) mice. After 21 days, tumor growth is generally established and the effects of the test agent after this point can be compared to vehicle-alone.

Breast cancer: Breast cancer cell lines such as MDA-MB-231 are injected preferably into the left cardiac ventricle of mice. Many breast cancers metastasize to bone. About 4 weeks after inoculation, tumors and bone metastases can be evaluated as can the effect of administration of the test agent.

Squamous cell carcinoma: Human basaloid squamous cell carcinoma cells or established tumor lines such as HTB-1 are administered either subcutaneously or submucosally into mice. After allowing a sufficient time for primary tumor growth, the mice are administered test or control preparations, and the effects of the test agent on the parameters described above are determined.

Other transplantable cell lines useful in these assays include, but are not limited to, human NCI-H522 lung tumor cell line (nude mice recipients), human SKOV3 cell line, and the M5076 cell line.

Cell Culture Assays: Primary cultures of HREC are prepared and maintained in passage 3-6 can be used for these studies. The identity of endothelial cells in cultures is validated by demonstrating endothelial cell incorporation of fluorescent-labeled acetylated LDL and by flow cytometry analysis as previously described. To maintain purity of HREC, several precautionary steps can be taken. HREC are grown in plasma-derived serum, which is free of platelet derived growth factor and does not promote the growth of pericytes. In addition, cultures of HREC are exposed to trypsin for only 45 sec prior to passage. Endothelial cells float off during this short trypsin treatment while pericytes remain attached to the substrate.

Proliferation Assay: HREC are seeded at 10 cells/cm in 24 well plates and allowed to adhere overnight. Cells are washed in Hank's balanced salt solution and the medium is replaced with serum—and growth supplement-free medium (SFM) for 24 hr to induce cell-cycle arrest. Cells are washed again and pre-treated with the compounds. Controls are HREC exposed to SFM or normal growth medium. For the next three days at 24-hour intervals replicate wells are treated with trypsin and the cells are collected and counted using a Coulter Counter. Each condition is examined in triplicate in three separate experiments using cells from different donors for each experiment.

Chemotaxis: Endothelial cell chemotaxis is measured in blind-well chemotaxis chambers (Neuroprobe, Inc, Bethesda, Md.). Briefly a single cell suspension of endothelial cells (1.0×10²-1×10⁶ cells/well) are prepared and treated with each compound. Thirty microliters of this suspension is placed in each of 48 lower wells of the blind-well apparatus. The wells are overlaid with a porous (5 μm diameter pore) polyvinyl—and pyrrolidone-free polycarbonate membrane (Nucleopore, Pleasanton, Calif.), coated with 0.1% dermal collagen. The cells were allowed to attach to the membrane by inverting the chamber of 2 h. The chambers were then placed upright and each exposed to the compounds (included are all the appropriate controls) in a 50 μL volume. After incubation for 12 h, the membrane is recovered and scraped free of cells on the attachment side. T he remaining cells, those that migrate through the pores, were fixed in methanol, is stained with modified Wright's stain and then counter-stained with haematoxylin and eosin. T he positive control was 10% fetal bovine serum and the negative control was 1% ablumin. Chemokinesis, the non-oriented increase in cell migration in response to a stimulus, was measured by adding equal concentration of NECA or NECA plus one of the antagonists to both lower and upper chambers. Treatment conditions were examined in triplicate in three separate experiments.

Matrigel Assay: Endothelial tube formation is assessed on Matrigel. Briefly, Matrigel is thawed and kept at 4° C. Multi-well plates and pipette tips are chilled to −20° C. and Matrigel (125 μL) iss added to each well of a 48-well plate and allowed to harden for a minimum of 1 h at 37° C. HREC is dissociated enzymatically (2 min at 37° C. in 0.25% Trypsin-EDTA), centrifuged (300×g, 5 min) and re-suspended in serum-free media. Test agents (100 μL) are prepared at 2× final concentration and 100 μL are added to wells. HREC (1×10²-1×10⁶ in 100 μL per well) are then added and plates are incubated at 37° C. Wells are photographed 48 h after plating. Identical fields in each well are photographed to minimize the possible variation due to variable cell density caused by the settling of cells. Photographs are digitized and image analysis software (Scion Image) is used to measure total tube length in a predefined, comparable area from each well. All conditions are tested in duplicate or triplicate wells in three separate experiments using cells from different donors.

Pharmaceutical Compositions

Dosage forms containing compounds of Formula I and/or II as the active ingredient may be advantageously used to treat pathological conditions involving angiogenesis. The dosage forms may be administered or applied singly, or in combination with other agents. The formulations may also deliver a compound of Formula I and/or II to a patient in combination with another pharmaceutically active agent, including another compound of Formula I and/or II. While it is possible for an active ingredient to be administered alone, it is preferable to present it as a formulation comprising an active ingredient in association with a pharmaceutically acceptable carrier therefor and, optionally, other therapeutic ingredient(s). The carrier(s) must be “acceptable” in the sense of being compatible with the other ingredients of the formulations and not deleterious to the recipient thereof.

In embodiments of the invention involving methods for stimulation of angiogenesis by administration of an anabaseine agonist, a compound of Formula I and/or II may be advantageously combined with agents that enhance angiogenesis by enhancing nitric oxide (NO) levels (e.g., by enhancing activity of NO synthase, by enhancing release of NO, etc.) or prostacyclin levels (e.g., by enhancing activity of prostacyclin synthase, by enhancing release of prostacyclin, etc.). Exemplary NO level-enhancing agents include, but are not necessarily limited to, L-arginine, L-lysine, and peptides enriched with these amino acids which can serve as substrates for NO; agents that preserve NO activity such as antioxidants (e.g., tocopherol, ascorbic acid, ubiquinone) or antioxidant enzymes (e.g., superoxide dismutase); and agents which can enhance NO synthase activity (e.g., tetrahydrobiopterin, or precursors for tetrahydrobiopterin (e.g., sepiapterin)); and the like. Exemplary prostacyclin level-enhancing agents include, but are not limited to precursors for prostacyclin such as eicosopentanoic acid and docosohexanoic acid; and prostanoids such as prostaglandin El and its analogues; and the like.

Alternatively or in addition, the pharmaceutical compositions according to the invention can comprise additional angiogenesis-inducing and/or vasculogenesis inducing agents that act through pathways other than the nicotine receptor (e.g., VEGF, FGF (e.g., aFGF, bFGF), Dell, etc.).

Formulations of the present invention suitable for oral administration may be in the form of discrete units such as capsules, cachets, tablets, or lozenges, each containing a predetermined amount of the active ingredient; in the form of a powder or granules; in the form of a solution or a suspension in an aqueous liquid or nonaqueous liquid; or in the form of an oil-in-water emulsion or a water-in-oil emulsion. The active ingredient may also be in the form of a bolus, electuary, or paste.

A tablet may be made by compressing or molding the active ingredient optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispensing agent. Molded tablets may be made by molding, in a suitable machine, a mixture of the powdered active ingredient and a suitable carrier moistened with an inert liquid diluent.

Formulations suitable for parenteral administration conveniently comprise a sterile aqueous preparation of the active ingredient which is preferably isotonic with the blood of the recipient and optional suitable excipients.

The pharmacologically active compounds of Formula I and/or II are useful in the manufacture of pharmaceutical compositions comprising an effective amount thereof in conjunction or admixture with the excipients or carriers suitable for either enteral or parenteral application. Preferred are tablets and gelatin capsules comprising the active ingredient together with one or more of the following: (a) diluents, such as lactose, dextrose, sucrose, mannitol, sorbitol, cellulose, glycine and the like; (b) lubricants, such as silica, talcum, stearic acid, its magnesium or calcium salt, polyethyleneglycol and the like; for tablets also; (c) binders, such as magnesium aluminum silicate, starch paste, gelatin, tragacanth, methylcellulose, sodium carboxymethyl-cellulose or polyvinylpyrrolidone and the like; and, if desired, (d) disintegrants, such as effervescent mixtures and the like; and (e) absorbents, colorants, flavors, and sweeteners and the like. Injectable compositions are preferably aqueous isotonic solutions or suspensions, and suppositories are advantageously prepared from fatty emulsions, or suspensions. Said pharmaceutical compositions may be sterilized and/or contain adjuvants, such as preserving, stabilizing, wetting or emulsifying agents, solution promoters, salts for regulating the osmotic pressure, and/or buffers. In addition, they may also contain other therapeutically valuable substances. Said compositions are prepared according to conventional mixing, granulating, or coating methods, respectively, and contain about 0.1 to 75%, preferably about 1 to 50%, of the active ingredient.

The formulations may conveniently be presented in unit dosage form and may be prepared by any of the methods well-known in the art of pharmacy. All methods include the step of bringing the active ingredient into association with the carrier which constitutes one or more accessory ingredients. In general, the formulations are prepared by uniformly and intimately bringing the active ingredient into association with a liquid carrier or a finely divided solid carrier or both, and then, if necessary, shaping the product into the desired formulation.

For certain embodiments of the invention, such as enhancement of wound or ulcer healing, topical dosage forms are preferred. Suitable formulations and dosage forms include ointments, creams, gels, lotions, pastes, and the like. Ointments, as is well known in the art of pharmaceutical formulation, are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. (See; Remington; The Science and Practice of Pharmacy, 19th Ed., Easton, Pa., Mack Publishing Co., 1995, pp. 1399-1404.)

Creams are viscous liquids or semisolid emulsions, either oil-in-water or water-in-oil. Cream bases are water-washable, and contain an oil phase, an emulsifier and an aqueous phase. The oil phase, also called the “internal’ phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Gels are semisolid, suspension-type systems. Single-phase gels contain organic macromolecules distributed substantially uniformly throughout the carrier liquid, which is typically aqueous, but also, preferably, contain an alcohol such as ethanol or isopropanol and, optionally, an oil. Preferred organic macromolecules i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose: gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions are preparations to be applied to the skin surface without friction, and are typically liquid or semi liquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylamnionium (DOTMA) liposomes are available under the trade name Lipofectin® (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art as comprised of surfactant molecules arranged so that their polar head groups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethyiammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface. Microspheres, similarly, may be incorporated into the formulations of the present invention and drug delivery systems. Like liposomes and micelles, microspheres essentially encapsulate a drug or drug-containing formulation. They are generally although not necessarily formed from lipids, preferably charged lipids such as phospholipids. Preparation of lipidic microspheres is well known in the art and described in the pertinent texts and literature.

The concentration of the active agent in the formulation can vary a great deal, and will depend on a variety of factors, including the disease or condition to be treated, the nature and activity of the active agent, the desired effect, possible adverse reactions, the ability and speed of the active agent to reach its intended target, and other factors within the particular knowledge of the patient and physician. The formulations will typically contain on the order of about 0.5 wt % to 50 wt % active agent, preferably about 0.5 wt % to 5 wt % active agent, optimally about 5 wt % to 20 wt % active agent.

Drug Delivery Systems

An alternative method of delivery of compositions of the invention involves the use of a drug delivery system, e.g., a topical or transdermal “patch,” wherein the active agent or agents are contained within a laminated structure that is to be affixed to the skin. Examples of suitable drug delivery systems are found in published patent applications no. WO 03/026654, no. JP 1717687, and no. JP 3460538, which are herein incorporated by reference. In such drug delivery systems, the drug composition is contained in a layer, or “reservoir,’ underlying an upper backing layer. The laminated structure may contain a single reservoir, or it may contain multiple reservoirs.

The reservoir may comprise a polymeric matrix of a pharmaceutically acceptable adhesive material that serves to affix the system to the skin during drug delivery; typically, the adhesive material is a pressure-sensitive adhesive that is suitable for long-term skin contact, and which should be physically and chemically compatible with the active agent(s), hydroxide-releasing agent, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene(polychloroprene).

The backing layer functions as the primary structural element of the transdermal system and provides the device with flexibility and, preferably, occlusivity. The material used for the backing layer should be inert and incapable of absorbing drug(s), hydroxide-releasing agent or components of the formulation contained within the device. The backing is preferably comprised of a flexible elastomeric material that serves as a protective covering to prevent loss of drug(s) and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. Immediately prior to use, this layer is removed from the device so that the system may be affixed to the skin. The release liner should be made from a drug/vehicle impermeable material, and is a disposable element which serves only to protect the device prior to application. Typically, the release liner is formed from a material impermeable to the pharmacologically active agent(s) and the hydroxide-releasing agent, and which is easily stripped from the transdermal patch prior to use.

In an alternative embodiment, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or ‘pouch,” or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a hydroxide-releasing agent, an additional enhancer, or some other component contained in the drug delivery system. A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5 cm² to 200 cm², preferably 5 cm² to 100 cm², more preferably 2 cm² to 60 cm². That area will vary, of course, with the amount of drug to be delivered and the flux of the drug through the body surface. Larger patches will be necessary to accommodate larger quantities of drug, while smaller patches can be used for smaller quantities of drug and/or drugs that exhibit a relatively high permeation rate. Such drug delivery systems may be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking’ in a drug/vehicle mixture. In general, transdermal systems of the invention are fabricated by solvent evaporation, film casting, melt extrusion, thin film lamination, die cutting, or the like. The hydroxide-releasing agent will generally be incorporated into the device during patch manufacture rather than subsequent to preparation of the device. In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such an adhesive overlayer, the delivery system remains in place for the required period of time.

Therapeutic Administration and Doses

The present methods for treating pathological conditions involving angiogenesis require administration of a compound of Formula I and/or II or a pharmaceutical composition containing a compound of Formula I and/or II, to a patient in need of such treatment. The compounds and/or pharmaceutical compositions are preferably administered orally. The compounds and/or pharmaceutical compositions may also be administered by any other convenient route, for example, by infusion or bolus injection, by absorption through epithelial or mucocutaneous linings (e.g., oral mucosa, rectal and intestinal mucosa, etc.). Administration can be systemic or local. Various delivery systems are known, (e.g., encapsulation in liposomes, microparticles, microcapsules, capsules, etc.) that can be used to administer a compound and/or composition. Methods of administration include, but are not limited to, intradermal, intramuscular, intraperitoneal, intravenous, intra-arterial, intrapericardial, subcutaneous, intranasal, epidural, oral, sublingual, intranasal, intracerebral, intravaginal, transdermal, rectally, by inhalation, or topically, particularly to the ears, nose, eyes, or skin.

The amount of a compound of Formula I and/or II that will be effective in a method for the treatment of pathological conditions involving angiogenesis in a patient will depend on the specific nature of the condition and can be determined by standard clinical techniques known in the art. In addition, in vitro or in vivo assays may optionally be employed to help identify optimal dosage ranges. The specific dose level for anyparticular individual will depend upon a variety of factors including the activity of the compound of Formula I and/or II, the age, body weight, general physical and mental health, genetic factors, environmental influences, sex, diet, time of administration, route of administration, rate of excretion, and the severity of the particular problem being treated.

Preferably, the dosage forms are adapted to be administered to a patient no more than twice per day, more preferably, only once per day. Dosing may be provided alone or in combination with other drugs and may continue as long as required for effective treatment of the pathological condition involving angiogenesis.

Suitable dosage ranges for oral administration are dependent on the potency of the particular compound of Formula I and/or II, but are generally about 0.001 mg to about 500 mg of drug per kilogram body weight, preferably from about 0.1 mg to about 200 mg of drug per kilogram body weight, and more preferably about 1 to about 100 mg/kg-body wt. per day. Dosage ranges may be readily determined by methods known to the skilled artisan. The amount of active ingredient that may be, for instance, combined with carrier materials to produce a single dosage form will vary depending upon the patient treated and the particular mode of administration. Dosage unit forms will generally contain between about 1 mg to about 500 mg of active ingredient.

Conditions Suitable for Treatment

The present invention provides methods for controlling angiogenesis in a patient in need thereof by administration of anabaseine agonists or anabaseine antagonists. These methods provide treatment for pathological conditions involving angiogenesis. In one embodiment, the methods and anabaseine agonist-comprising compositions of the invention can be used to treat a variety of conditions that would benefit from stimulation of angiogenesis, stimulation of vasculogenesis, increased blood flow, and/or increased vascularity. In another embodiment, the methods and anabaseine antagonist-comprising compositions of the invention can be used to treat any condition or disorder that is associated with or that results from pathological angiogenesis, or that is facilitated by neovascularization (e.g., a tumor that is dependent upon neovascularization).

Examples of conditions and diseases suitable for treatment according to the method of the invention include any condition associated with an obstruction of a blood vessel, e.g., obstruction of an artery, vein, or of a capillary system. Specific examples of such conditions or disease include, but are not necessarily limited to, coronary occlusive disease, carotid occlusive disease, arterial occlusive disease, peripheral arterial disease, atherosclerosis, myointimal hyperplasia (e.g., due to vascular surgery or balloon angioplasty or vascular stenting), thromboangitis obliterans, thrombotic disorders, vasculitis, ischemic limb disease, ischemic colitis, and the like. Examples of conditions or diseases that can be prevented using the methods of the invention include, but are not necessarily limited to, heart attack (myocardial infarction) or other vascular death, stroke, death or loss of limbs associated with decreased blood flow, and the like.

Other forms of therapeutic angiogenesis include, but are not necessarily limited to, the use of anabaseine agonists to accelerate healing of wounds or ulcers, such as those due to diabetic ischemia; to improve the vascularization of skin grafts or reattached limbs so as to preserve their function and viability; to improve the healing of surgical anastomosis (e.g., as in re-connecting portions of the bowel after gastrointestinal surgery); and to improve the growth of skin or hair.

Conditions and disorders suitable for treatment by methods involving anabaseine antagonists include, but are not limited to, cancer; atherosclerosis; proliferative retinopathies such as diabetic retinopathy, age-related maculopathy, retrolental fibroplasia; excessive fibrovascular proliferation as seen with chronic arthritis; psoriasis; and vascular malformations such as hemangiomas, and the like. The instant methods are useful in the treatment of both primary and metastatic solid tumors, including carcinomas, sarcomas, leukemias, and lymphomas. Of particular interest is the treatment of tumors occurring at a site of angiogenesis. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The present methods are also useful for treating solid tumors arising from hematopoietic malignancies such as leukemias (i.e. chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy and/or other chemotherapeutic agents.

Other conditions and disorders amenable to treatment using the methods of the instant invention include autoimmune diseases such as rheumatoid, immune and degenerative arthritis; various ocular diseases such as diabetic retinopathy, retinopathy of prematurity, corneal graft rejection. retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemangiomas, and capillary proliferation within atherosclerotic plaques; Osler-Webber Syndrome; plaque neovascularization; telangiectasia; hemophiliac joints; angiofibroma; and excessive wound granulation (keloids).

Combination Therapy

In another preferred embodiment, compounds of the invention can be administered with one or more other therapeutic agent such as angiogenesis inhibitors. Examples include, but not limited to: antiestrogens, progestogens, aromatase inhibitors, antihormones, antiprogestogens, antiandrogens, LHRH agonists and antagonists, testosterone 5α-dihydroreductase inhibitors, farnesyl transferase inhibitors, anti-invasion agents, growth factor inhibitors, antimetabolites, intercalating antitumour antibiotics, platinum derivatives, alkylating agents, antimitotic agents, topoisomerase inhibitors, cell cycle inhibitors, and biological response modifiers, linomide, integrin αvβ3 function inhibitors, angiostatin, razoxin, tamoxifen, toremifen, raloxifene, droloxifene, iodoxyfene, megestrol acetate, anastrozole, letrazole, borazole, exemestane, flutamide, nilutamide, bicalutamide, cyproterone acetate, gosereline acetate, luprolide, finasteride, metalloproteinase inhibitors, urokinase plasminogen activator receptor function inhibitors, growth factor antibodies, growth factor receptor antibodies, tyrosine kinase inhibitors, serine/threonine kinase inhibitors, methotrexate, 5-fluorouracil, purine, adenosine analogues, cytosine arabinoside, doxorubicin, daunomycin, epirubicin, idarubicin, mitomycin-C, dactinomycin, mithramycin, cisplatin, carboplatin, nitrogen mustard, melphalan, chlorambucil, busulphan, cyclophosphamide, ifosfamide nitrosoureas, thiotephan, vincristine, taxol, taxotere, epothilone analogs, discodermolide analogs, eleutherobin analogs, etoposide, teniposide, amsacrine, topotecan, flavopyridols, and biological response modifiers. In some preferred embodiments, the additional thereapeutic agent is selected from Erbitux™, taxol, paraplatin and Ifex.

The compositions and methods of the invention in certain instances may be useful for replacing existing surgical procedures or drug therapies, although in most instances the present invention can also improve the efficacy of existing therapies for treating such conditions. Accordingly combination therapy may be used to treat the subjects. For example, the agent may be administered to a subject in combination with another anti-proliferative (e.g., an anti-cancer) therapy. Suitable anti-cancer therapies include surgical procedures to remove the tumor mass, chemotherapy or localization radiation. The other anti-proliferative therapy may be administered before, concurrent with, or after treatment with the agent of the invention. There may also be a delay of several hours, days and in some instances weeks between the administration of the different treatments, such that the agent may be administered before or after the other treatment.

As an example, the agent may be administered in combination with surgery to remove an abnormal proliferative cell mass. As used herein, “in combination with surgery” means that the agent may be administered prior to, during or after the surgical procedure. Surgical methods for treating epithelial tumor conditions include intra-abdominal surgeries such as right or left hemicolectomy, sigmoid, subtotal or total colectomy and gastrectomy, radical or partial mastectomy, prostatectomy and hysterectomy. In these embodiments, the agent may be administered either by continuous infusion or in a single bolus. Administration during or immediately after surgery may include a lavage, soak or perfusion of the tumor excision site with a pharmaceutical preparation of the agent in a pharmaceutically acceptable carrier. In some embodiments, the agent is administered at the time of surgery as well as following surgery in order to inhibit the formation and development of metastatic lesions. The administration of the agent may continue for several hours, several days, several weeks, or in some instances, several months following a surgical procedure to remove a tumor mass.

The subjects can also be administered the agent in combination with non-surgical anti-proliferative (e.g., anti-cancer) drug therapy. In one embodiment, the agent may be administered in combination with an anti-cancer compound such as a cytostatic compound. A cytostatic compound is a compound (e.g., a nucleic acid, a protein) that suppresses cell growth and/or proliferation. In some embodiments, the cytostatic compound is directed towards the malignant cells of a tumor. In yet other embodiments, the cytostatic compound is one which inhibits the growth and/or proliferation of vascular smooth muscle cells or fibroblasts.

Suitable anti-proliferative drugs or cytostatic compounds to be used in combination with the agents of the invention include anti-cancer drugs. Anti-cancer drugs are well known and include: Acivicin; Aclarubicin; Acodazole Hydrochloride; Acronine; Adozelesin; Aldesleukin; Altretamine; Ambomycin; Ametantrone Acetate; Aminoglutethimide; Amsacrine; Anastrozole; Anthramycin; Aspariaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene: Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estrarnustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Taxotere; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-cancer drugs include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; z7azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+ mycobacterium cell wall sk; mopidamol; multiple drug resistance gene inhibitor; multiple tumor suppressor 1-based therapy; mustard anti cancer compound; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotidc; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists;. raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizoftran.; sobuzoxane; sodium borocaptate; sodium phenylacetate; solverol; somatomedin binding protein; sonermin; sparfosic acid; spicamycin D; spiromustinc; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

Anti-cancer supplementary potentiating compounds include: Tricyclic anti-depressant drugs (e.g., imipramine, desipramine, amitryptyline, clomipraminc, trimipramine, doxepin, nortriptyline, protriptyline, amoxapine and maprotiline); non-tricyclic anti-depressant drugs (e.g., sertraline, trazodone and citalopram); Ca⁺⁺ antagonists (e.g., verapamil, nifedipine, nitrendipine and caroverine); Calmodulin inhibitors (e.g., prenylamine, trifluoroperazine and clomipramine); Amphotericin B; Triparanol analogues (e.g., tamoxifen); antiarrhythmic drugs (e.g., quinidine); antihypertensive drugs (e.g., reserpine); Thiol depleters (e.g., buthionine and sulfoximine) and multiple drug resistance reducing compounds such as Cremaphor EL.

Other compounds which are useful in combination therapy for the purpose of the invention include the antiproliferation compound, Piritrexim Isethionate; the antiprostatic hypertrophy compound, Sitogluside; the benign prostatic hyperplasia therapy compound, Tamsulosin Hydrochloride; the prostate growth inhibitor, Pentomone; radioactive compounds such as Fibrinogen I¹²⁵, Fludeoxyglucose F¹⁸, Fluorodopa F¹⁸, Insulin ¹²⁵I, Insulin ¹³¹I, lobenguane ¹²³I, lodipamide Sodium ¹³¹I, lodoantipyrine ¹³¹I, lodocholesterol ¹³¹I, lodohippurate Sodium ¹²³I, lodohippurate Sodium ¹²⁵I, iodohippurate Sodium ¹³¹I, lodopyracet ¹²⁵I, lodopyracet ¹³¹I, Iofetamine Hydrochloride ¹²³I, lomethin ¹²⁵I, lomethin ¹³¹I, lothalamate Sodium ¹²⁵I, lothalamatc Sodium ¹³¹I, lotyrosine ¹³¹I, Liothyronine ¹²⁵I, Liothyronine ¹³¹I, Merisoprol Acetate ¹⁹⁷Hg, Mcrisoprol Acetate ²⁰³Hg, Merisoprol ¹⁹⁷Hg, Selenomethionine 75^{Se}, Technetium Tc ^{99m} Antimony Trisulfide Colloid, Technetium ^{99m}Tc Bicisate, Technetium ^{99m}Tc Disofenin, Technetium ^{99m}Tc Etidronate, Technetium ^{99m}Tc Exametazime, Technetium ^{99m}Tc Furifosmin, Technetium ^{99m}Tc Gluceptate, Technetium ^{99m}Tc Lidofenin, Technetium ^{99m}Tc Mebrofenin, Technetium ^{99m}Tc Medronate, Technetium ^{99m}Tc Medronate Disodium, Technetium ^{99m}Tc Mertiatide, Technetium ^{99m}Tc Oxidronate, Technetium ^{99m}Tc Pentetate, Technetium ^{99m}Tc Pentetate Calcium Trisodium, Technetium ^{99m}Tc Sestamibi, Technetium ^{99m}Tc Siboroxime, Technetium ^{99m}Tc Succimer, Technetium ^{99m}Tc Sulfur Colloid, Technetium ^{99m}Tc Teboroxime, Technetium ^{99m}Tc Tetrofosmin, Technetium ^{99m}Tc Tiatide, Thyroxine ¹²⁵I, Thyroxine ¹³¹I, Tolpovidone ¹³¹I, Triolein ¹²⁵I and Triolein ¹³¹I.

According to the methods of the invention, the agents of Formula I and/or II may be administered prior to, concurrent with, or following the other anti-cancer compounds. The administration schedule may involve administering the different agents in an alternating fashion. In other embodiments, the agent may be delivered before and during, or during and after, or before and after treatment with other therapies. In some cases, the agent is administered more than 24 hours before the administration of the other anti-proliferative treatment. In other embodiments, more than one anti-proliferative therapy may be administered to a subject. For example, the subject may receive the agents of the invention, in combination with both surgery and at least one other anti-proliferative compound. Alternatively, the agent may be administered in combination with more than one anti-cancer drug.

Other compounds useful in combination therapies with the inhibitor compounds of the invention include anti-angiogenic compounds such as angiostatin, endostatin, fumagillin, non-glucocorticoid steroids and heparin or heparin fragments and antibodies to one or more angiogenic peptides such as αFGF, βFGF, VEGF, IL-8 and GM-CSF. These latter anti-angiogenic compounds may be administered along with the inhibitor agents of the invention (i.e., the agents of Formula I and/or II) for the purpose of inhibiting proliferation or inhibiting angiogenesis in all of the aforementioned conditions as described herein. In certain embodiments, the agent may be administered in combination with an anti-angiogenic compound and at least one of the anti-proliferative therapies described above including surgery or anti-proliferative drug therapy.

Other examples include, but not limited to: endostatin, chemokines, angioarrestin, angiostatin (plasminogen fragment), anti-angiogenic antithrombin III, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-b), vasculostatin, vasostatin (calreticulin fragment).

The above-described drug therapies are well known to those of ordinary skill in the art and are administered by modes known to those of skill in the art. The drug therapies are administered in amounts which are effective to achieve physiological goals such as the inhibition of proliferation or inhibition of angiogenesis, in combination with the agents of the invention. It is contemplated that the drug therapies may be administered in amounts which, when used alone, may not be capable of inhibiting proliferation or angiogenesis but which, when administered in combination with the agents of the invention, are capable of achieving the desired level of inhibition. Thus, in embodiments in which the agent of Formula I and/or II is administered with another therapeutic agent (e.g., an anti-proliferative compound or an anti-angiogenic compound), subtherapeutic doses of either or both agents may be used. In still other embodiments, anti-proliferative drug therapies may be administered in conditions such as doses or amounts which do not affect hemopoietic cell levels in the subjects.

The agents of the invention are administered in therapeutically effective amounts. An effective amount is a dosage of the agent sufficient to provide a medically desirable result. The effective amount will vary with the particular condition being treated, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, in connection with methods directed towards treating subjects having a condition characterized by abnormal mammalian cell proliferation, an effective amount to inhibit proliferation would be an amount sufficient to reduce or halt altogether the abnormal mammalian cell proliferation so as to slow or halt the development of or the progression of a cell mass such as, for example, a tumor. As used in the embodiments, “inhibit” embraces all of the foregoing.

According to other aspects of the invention directed at inhibiting angiogenesis in a subject having a condition characterized by an abnormal mammalian cell proliferation, an effective amount to inhibit angiogenesis would be an amount sufficient to lessen or inhibit altogether smooth muscle cell proliferation so as to slow or halt the development of or the progression of tumor vascularization. As used in these embodiments, “inhibit” embraces all of the foregoing.

The “treatment of cancer or tumor cells”, refers to an amount of compounds of Formula I and/or II, described throughout the specification and in the Examples which follow, capable of invoking one or more of the following effects: (1) inhibition, to some extent, of tumor growth, including, (i) slowing down (ii) inhibiting angiogenesis and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

When used therapeutically, the agent is administered in therapeutically effective amounts. In general, a therapeutically effective amount means that amount necessary to delay the onset of, inhibit the progression of, or halt altogether the particular condition being treated. In some aspects of the invention, an therapeutically effective amount will be that amount necessary to inhibit mammalian cell proliferation. In other embodiments, a therapeutically effective amount will be an amount necessary to extend the dormancy of micrometastases or to stabilize any residual primary tumor cells following surgical or drug therapy.

In still other embodiments, the agent is delivered in an amount, a dose, and a schedule which is therapeutically effective in inhibiting proliferation yet which is not therapeutically effective in stimulating hemopoiesis in the subject. In administering the agents of the invention to subjects, dosing amounts, dosing schedules, routes of administration and the like can be selected so as to affect the other known activities of these compounds. For example, amounts, dosing schedules and routes of administration can be selected as described below, whereby therapeutically effective levels for inhibiting proliferation are provided, yet therapeutically effective levels for restoring hemopoietic deficiency are not provided. As another example, local administration to tumors or protected body areas such as the brain may result in therapeutically effective levels for inhibiting proliferation, but may be non-therapeutically effective levels for hemopoietic cell stimulation.

In addition, agents of Formula I and/or II can be selected that are effective as anti-proliferative agents but are relatively ineffective as hemopoietic cell stimulatory or activating agents. Thus, certain subjects who require both hemopoietic stimulation and/or activation and proliferation inhibition may be treated with different agents of Formula I and/or II simultaneously, one each for the desired therapeutic effect, or with a single agent but in different dosages, schedules, and/or route to achieve both hemopoietic stimulation and proliferation inhibition at therapeutic levels.

Generally, a therapeutically effective amount will vary with the subject's age, condition, and sex, as well as the nature and extent of the disease in the subject, all of which can be determined by one of ordinary skill in the art. The dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days. In some embodiments, the agents are administered for more than 7 days, more than 10 days, more than 14 days and more than 20 days. In still other embodiments, the agent is administered over a period of weeks, or months. In still other embodiments, the agent is delivered on alternate days. For example, the agent is delivered every two days, or every three days, or every four days, or every five days, or every six days, or every week, or every month.

The agents of the invention can also be administered in prophylactically effective amounts, particularly in subjects diagnosed with benign or pre-malignant tumors. In these instances, the agents are administered in an amount effective to prevent the development of an abnormal mammalian cell proliferative mass or to prevent angiogenesis in the solid tumor mass, depending on the embodiment. The agents may also be administered in an amount effective to prevent metastasis of cells from a tumor to other tissues in the body. In these latter embodiments, the invention is directed to preventing the metastatic spread of a primary tumor.

Determination of Therapeutic Effectiveness

The effectiveness of a compound of Formula I and/or II for its intended use in a method of the invention can be determined in a variety of ways. For example, compounds may be selected for further testing in the methods of the invention on the basis of data from in vitro assays and/or in vivo animal models.

Whether a compound of Formula I and/or II functions as an anabaseine agonist or an anabaseine antagonist can be determined by in vitro assays known to those skilled in the art. Binding of a putative anabaseine agonist or anabaseine antagonist to mammalian brain nicotine acetylcholine receptor (nAChR) can be measured, for example, by the method of W. R. Kem et al). (Mol. Pharm., 2004, 65: 56-67) via a radioligand assay. Binding at the α7 nAChR is determined by competitive inhibition of the specific binding of ¹²⁵I-bungarotoxin. By binding is meant having a Kᵢ value of about ≦10⁻³ M to about ≦10⁻¹¹ M, preferably about ≦10⁻⁴ M to about −<10¹⁰ M, or a K_{d} value of about ≦10⁻³ M to about ≦10⁻¹¹ M, preferably about ≦10⁻⁴ M to about ≦10¹⁰ M. After assuring that a compound of Formula I and/or II binds to a nicotine acetylcholine receptor, and preferably binds to an α7 nAChR, efficacy studies for the determination of agonism vs. antagonism at a receptor can be made by the method of Kem et al. (2004) using rodent and human nicotinic receptors expressed in X. laevis oocytes. Agonists have EC₅₀ values of about ≦0.1 μM to about ≦500 μM, preferably about ≦1 μM to about ≦200 μM. Partial agonists have EC₅₀ values of about ≦0.1 μM to about ≦500 μM, preferably about ≦1 μM to about ≦200 μM. Antagonists have EC₅₀ values of about ≦10⁻³ M to about ≦10⁻¹¹ M, preferably about ≦10⁻⁴ M to about ≦10⁻¹⁰ M. Selection of anabaseine agonists or anabaseine antagonists for further testing in the control of angiogenesis can be made on the basis of such binding and efficacy studies.

The ability of compounds of Formula I and/or II to induce or enhance angiogenesis/vasculogenesis may be tested by a variety of methods known to one skilled in the art. Such methods include, but are not limited to, stimulation of angiogenesis in the disc angiogenesis system (DAS) (e.g., Kowalski et al, Exp. Mol. Pathol, 56(1 ):1-1 9; Fajardo et al., Lab. Invest, 1998, 58:718-724), enhancement of in vitro calf pulmonary artery endothelial cells (e.g., A. C. Villablanca, J. Appl. Physiol, 1998, 84(6): 2089-2098), improvement of wound healing in genetically diabetic BKS.Cg-m^{+/+} Lepr^{db} mice (e.g., J. Jacobi et al, Am. J Path., 2002, 161(1): 97-104), induction of neovascularization in a Nos3^{-/-} mouse model of hind-limb ischemia (e.g., A. Aicher et al, Nature Med., 2003, 9(11):1370-1376) and enhancement of wound healing in normal mice (e.g., Canapp et al., Vet Surg. 2003, 32(6):515-23).

The ability of compounds of Formula I and/or II to reduce or inhibit angiogenesis may be tested by a variety of methods known to one skilled in the art. Such methods include, but are not limited to, inhibition of neovascularization into implants impregnated with an angiogenic factor; inhibition of blood vessel growth in the cornea or anterior eye chamber; inhibition of endothelial cell proliferation, migration or tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al. (Pharmac. Ther., 1991, 51:1-11), and references cited therein.

The invention further provides methods for treating a condition or disorder associated with or resulting from pathological angiogenesis. In the context of cancer therapy, a reduction in angiogenesis according to the methods of the invention results in a reduction in tumor size and/or a reduction in tumor metastasis. Animal models for testing the reduction of angiogenesis of tumors include, but are not limited to, the Lewis Lung tumor model (e.g., C. H. Heeschen et al, J. Clin. Invest., 2002, 110:527-536) and the Caki-1 model in mice (e.g., D. W. Siemann and A. M. Rojiani, Int. Radiat Oncol. Biol. Phys., 2002, 54(5):1 512-7). In mammals, and preferably in humans, whether a reduction in tumor size is achieved can be determined, for example, by measuring the size of the tumor, using standard imaging techniques. Whether metastasis is reduced can be determined using methods known to those skilled in the art. Methods to assess the effect of an agent on tumor size are well known and include imaging techniques such as computerized tomography and magnetic resonance imaging.

The efficacy of the methods and compositions of the present invention in the control of angiogenesis can also optionally be evaluated in human clinical trials conducted under appropriate standards and ethical guidelines. Additionally, the state of the disease before and after treatment may be assessed by various commonly accepted examinations.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

The following examples are offered by way of illustration and not by way of limitation.

### EXAMPLES

Materials and Methods. Melting point (m.p.) values are uncorrected. Elemental analyses were supplied by Atlantic Microlabs, Inc. (Norcross, Ga.). 1H NMR chemical shifts (in ppm) were recorded in dimethyl sulfoxide-d₆ using tetramethylsilane as a reference on a VXR 300 spectrometer (Varian, Inc., Palo Alto, Calif.). Mass spectra of the synthetic compounds were recorded on an MS8ORFA spectrometer (Kratos Analytical, Manchester, UK). Fast atom bombardment (FAB) analyses used an 8-kV xenon beam and 3-nitrobenzyl alcohol as a matrix. Accurate mass measurements were carried out after direct introduction and 70-electron volt electron ionization at a nominal mass resolution of 10,000 with a perfluorokerosene internal standard.

Radioligands [³H]cytisine and [¹²⁵I]BTX were purchased from PerkinElmer Life and Analytical Sciences (Boston, Mass.). Reagents for chemical synthesis and various solvents were purchased from Sigma-Aldrich (St. Louis, Mo.). Commonly used chemicals and solvents were purchased from Fisher Chemical Co. (Orlando, Fla.). Neuronal nAChR subunit cDNAs were graciously provided by Dr. James Boulter (Salk Institute, San Diego, Calif.) and Dr. Jon Lindstrom (Univ. of Pennsylvania, Philadelphia, Pa.). RNA transcription kits were purchased from Ambion (Austin, Tex.). Male Sprague Dawley rats (200-250 g) were purchased from Charles River Breeding Laboratories (Raleigh, N.C.) and Harlan (Indianapolis, Ind.).

### Example 1

### Synthesis of Selected Anabaseine Agonists and Antagonists

DMXBA, 3-[2,4-dimethoxy)benzylidene)-anabaseine (015-21). Synthesis of DMXBA was initiated by dissolving anabaseine dihydrochloride (6.09, 0.024 mol) and 2,4-dimethoxybenzaldehyde (9.0 g, 0.054 mol) in 350 ml of ethanol containing approximately 20 drops of conc. HCl. T he solution was stirred at 75° C. overnight. After cooling, 1.2 g of product was collected by filtration. Ether (approx. 800 ml) was added to the mother liquor until no more precipitate appeared. The combined solids were dissolved in methanol and precipitated with ether. This procedure was repeated several times. The product (8.1 g) was obtained as a yellow solid in 89% yield, m.p. 216-217° C. (dec). Analytical calculation for C₁₉H₂₀N₂O₂×2HCl (mol. wt. 381): C,59.85; H, 5.82; N, 7.35. Found: C, 59.54; H, 5.89; N, 7.41. ¹H NMR: δ 8.94 (d, J=1.5 Hz, H2′), 8.92 (dd, J=4.9 and 1.7 Hz, H6′), 8.22 (dd, J=8.6 and 1.9 Hz, H4′), 7.79 (dd, J=7.9 and 5.1 Hz, H5′), 7.60 (d, J=8.8 Hz, H13), 7.31 (s, H10), 6.70 (dd, J=8.8 and 2.4 Hz, H12), 6.65 (s, H7), 3.85 (s, OMe), 3.80 (t, J=5.7 Hz, H6), 3.72 (s, OMe), 2.92 (t, J=5.7 Hz, H4), and 2.02 (qn, J=5.6 Hz, H5). High-resolution electron ionization-MS: 308.1525 expected, 308.1557 calculated.

4-OH-MBA, 3-[4-hydroxy-2-methoxy)benzylidene]-anabaseine (GTS-62). 4-OH-MBA was prepared as a yellow hygroscopic solid in 85% yield from anabaseine hydrochloride and 4-hydroxy-2-methoxybenzaldehyde using the same procedure as described above, m.p. 215-219° C. (dec). Analytical calculation for C₁₈H₁₈N₂O₂×2HCl (mol. wt. 367): C, 58.87; H. 5.49; N, 7.63. Found: C, 58.40; H, 5.79; N, 6.33. ¹H NMR: δ 8.92 (d, J=5.0 Hz, H6′), 8.87 (d, J=1.8, H2′), 8.17 (d, J=8.0 Hz, H4′), 7.76 (dd, J=8.0 and 5.0 Hz, H5′), 7.55 (d, J=8.8 Hz, H13), 7.34 (s, H7), 6.59 (d, J=10.7 Hz, H12), 6.50 (d, J=2.3 Hz, H10), 3.78 (tr, J=10.7 Hz, H6), 3.65 (s, OMe), 2.93 (tr, J=11.1 Hz, H4), and 2.03 (tr, J=10.5 Hz, H5). FAB-MS: m/z 295 (M+H)⁺.

2-OH-MBA, 3-[2-hydroxy-4-methoxy)benzylidene]-anabaseine (GTS-51). 2-OH-MBA was prepared as a yellow hygroscopic solid in 86% yield from anabaseine hydrochloride and 2-hydroxy-4-methoxybenzaldehyde using the same procedure as described above, np. 215-219° C. (dcc). Analytical calculation for C₁₈H₁₈N₂O₂×2HCL (mol. wt. 367): C, 58.87; H, 5.49; N, 7.63. Found: C, 58.59; H, 5.76; N, 7.47. ¹H NMR: δ 8.82 (s, H2′), 8.80 (bd, J=4.9, H6′) 8.10 (dd, J=7.5 and 1.5 Hz, H4′), 7.71 (dd, J=7.5 and 4.5 Hz, H5′), 7.59 (d, J=9.0 Hz, H13), 7.41 (s, H7), 6.58 (d, J=8.6 Hz, H12), 6.52 (s, H10), 3.61 (tr, J=5.5 Hz, H6), 3.77 (s, OMe), 2.94 (tr, J=5.2 Hz, H4), and 2.03 (qn, J=5.1 Hz, H5). FAB-MS: m/z 295 (M+H)⁺.

2,4-OH—BA, 3-[2.4-dihydroxy)benzylidene]-anabaseine (GTS-52). 2,4-OH—BA was synthesized essentially as described above, but the acid catalyst was omitted because of the acid instability of 2,4-dihydroxybenzaldehyde. The product was obtained in 50% yield as a very hygroscopic solid, m.p. 256-259° C. (dec). Analytical calculation for C₁₇H₁₆N₂O₂×2HCL (mol. wt. 353); C, 57.80; H, 5.14; N, 7.93. Found: C, 56.71; H, 5.26; N, 7.77. ¹H NMR: 5 8.86 (bd, J=5.1, H6′), 8.79 (bs, H2′), 8.05 (d, J=7.9 Hz, H4′), 7.66 (dd, J=7.7 and 4.9 Hz, H5′) 7.51 (d, J=9.0 Hz, H13), 7.42 (s, H7), 6.39 (broad, H12 and N⁺H), 6.32 (s, H10), 3.78 (tr, J5.4 Hz, H6), 2.29 (tr, J=5.8 Hz, H4), and 2.02 (qn, J=5.1 Hz, H5). FAB-MS: m/z 281 (M+H)⁺.

3-F(4-Dimethylaminopropoxy)benzylidene]anabaseine (GTS-1 5). GTS-1 5 was synthesized essentially as described above, using the acid catalyst, by reaction of anabaseine dihydrochloride with 4-dimethylaminopropoxybenzaldehyde. ¹H-NMR (CDCl₃/TMS): δ 8.71 (H2′), 8.62 (H6′), 7.81 (H4′), 7.3 (H5′), 7.24, 6.87 (aromatic), 6.56 (vinyl), 4.03 (OCH₂), 3.86 (H6), 2.82 (H4), 2.50 (NCH₂), 2.23 (Me), 1.98 (CH₂), 1.82 (H5).

3-[(2,4-Dimethoxy)benzylidene]-6′-methyl anabaseine dihydrochloride (GTS-57). GTS-57 was synthesized essentially as described above with acid catalysis by reaction of 6′-Methylanabaseine dihydrochloride with dimethoxybenzaldehyde. This afforded the title compound as a yellow hygroscopic salt (90%). ¹H-NMR (DMSOITMS): δ 8.62 (s, H2′), 7.90 (d, J=0.3 Hz, H4′). 7.51 (d, J=8.1 Hz, H5′), 7.46 (d, J=8.7 Hz, H13), 7.12 (s, H7), 6.64 (d, J=8.90, H12), 6.60(s, H10), 3.82 (s, OMe), 3.75 (tr, J=5.3 Hz, H6), 3.70 (s, OMe), 2.82 (tr, J=5.1 Hz, H4), 1.90 (qn, J=5.2 Hz, H5), MS(FAB) (M+H)⁺ 322.

### Example 2

### Brain Nicotinic Receptor Radioligand Binding Assays

Washed whole rat brain membranes (200 pg of protein) were prepared according to the method used by Marks and Collins (Mol. Pharmacol., 1982, 22:554-564). Before use, the washed membranes were resuspended in 500-pt receptor binding assay saline (pH 7.40) consisting of 120 mM NaCl, 5 mM KCl, 2 mM CaCl₂, 1 mM MgCl₂ and 50 mM Tris-HCI. [³H]Cytisine (35 Ci/mmol)-binding displacement experiments were performed essentially according to Flores et al (1992), except that the incubation time was increased to 4 h at 0 to 4° C. to ensure equilibrium during the competition binding assay. Binding of ¹²⁵I-BTX (136 Ci/mmol) was performed at 37° C. for 3 h; the saline solution mentioned above also contained 2 mg/ml bovine serum albumin. Nonspecific binding of each radioligand was measured in the presence of 1.0 mM nicotine (Marks and Collins, 1982). After incubation, membranes with bound radioligand were collected on Whatman CF/C glass fiber filters presoaked for 45 mm in 0.5% polyethylenimine and washed three times with 3.0 ml of ice-cold buffer by vacuum filtration on a harvester (Brandel, Gaithersburg, Md.). Bound [³H]cytisine was measured with use of a Biogamma counter (both from Beckman Coulter). Binding data were analyzed using Prism software (GraphPad Software Inc., San Diego, Calif.). All Ki values were calculated from the Cheng-Prusoff equation, using a K_{d} value for each radioligand that had been experimentally determined under conditions identical with those of the displacement experiments.

The relative abilities of the compounds tested according to this method to interact with either rat brain α4β₂ or α7 nicotinic receptors are shown in Table 1. The concentration of each radioligand was 1 nM. Each value (in nM) represents the mean±S.E.M. of values obtained from four (BTX) or three (cytisine) separate experiments. The Kᵢ (Ion) values are inhibitory concentrations of the ionized form of the compound and were calculated assuming that only the cationic ionized form interacts with the ACh binding site. Because cytisine binds preferentially to the desensitized form of the α4β₂ receptor, which routinely displays a several-hundred-fold higher affinity for ACh than the resting form, whereas BTX binds preferentially to the resting state of the α7 receptor, these two sets of Kᵢ values are not comparable with each other because they were not obtained from equivalent states of these two receptors.

TABLE 1

| | ¹²⁵I-BTX Binding | | [³H] Cytisine Binding | |
|---|---|---|---|---|
| Compound | Kᵢ ± S.E.M. | Kᵢ₍Ion) | Kᵢ ± S.E.M. | Kᵢ₍Ion) |
| DMXBA | 130 ± 14 | 81 | 253 ± 37 | 158 |
| 4-OH-MBA | 235 ± 14 | 78 | 69 ± 30 | 23 |
| 2-OH-MBA | 317 ± 67 | 105 | 387 ± 25 | 128 |
| 2,4-OH-BA | 203 ± 5.0 | 102 | 206 ± 16 | 103 |

DMBXA competed with iodinated BTX for α7 binding sites and with tritiated cytisine for predominately α4β₂ receptor binding sites in rat brain in a manner consistent with competitive antagonism. The Scatchard plots (not shown) for DMXBA inhibition of receptor binding by these two radioligands showed significant changes in slope but no significant changes in Bₘₐₓ values. The mean Kd for ¹²⁵I-BTX binding, obtained from eight saturation experiments, was 0.32±0.04 nM. The Kᵢ calculated for DMXBA inhibition of iodinated BTX binding in these saturation experiments was 173 nM. 2-OH-MBA and 4-OH-MBA inhibited the specific binding of both cytisine and BTX in a like manner (data not shown). The affinities of the four compounds for the α7 receptor varied approximately 3-fold, with DMXBA showing the highest affinity for this nAChR subtype. When differences in ionization between compounds were accounted for, their affinities for this receptor varied less than 50%. Affinities for the α4β₂ receptor varied almost 4-fold, with 4-OH-MBA displaying the highest relative affinity. The predicted α4β₂ affinity of the ionized form of 4-OH-MBA was approximately 6-fold greater than for DMXBA monocation.

### Example 3

### Pharmacological Effects of DMXBA Metabolites Upon Rodent and Human Nicotine. Receptors Expressed in X. laevis Oocytes

The experimental protocols for obtaining concentration-response curves for the two metabolites, 2-OH-MBA and 2,4-OH—BA, were identical with those described previously for 4-OH-MBA and DMBXA (Papke and Porter-Papke, Br. J Pharmacol, 2002, 137:49-61). Peak currents were measured for responses of α4β₂ receptors to agonist application, because desensitization of this receptor subtype was slow enough not to affect the concentration-response relation measured in this manner. Currents resulting from agonist application to the more rapidly desensitizing α7 receptors were integrated over a period of 90× after application commenced. To compare data obtained from different oocytes, a standard response normalization procedure was used. Agonist responses of a particular oocyte were always normalized relative to its response to a standard acetylcholine (ACh) control. Before actual measurements of agonist properties, several ACh pulses separated by 5-mm intervals were applied until the responses of the oocyte to ACh became steady. After washing for 5 mm, the peak current response to the test compound was measured. After another 5-mm washing period, ACh was again applied to measure the residual responsiveness of the receptors to ACh after exposure to the test substance. The concentration-response data were fitted with a modified Hill equation, I=Iₘₐₓ [agonist]^{nH}/([agonist]^{nH}+EC₅₀), to estimate apparent efficacy (normalized maximal response) and EC₅₀.

The EC₅₀ values for the agonist effects of the four compounds tested on rat and human α7 nicotinic receptors are shown in Table 2 and show relatively minor differences. The numbers in parentheses in Table 2 are calculated EC₅₀, values for just the ionized forms. It is interesting that all three DMXBA hydroxyl metabolites exhibited efficacies that were consistently greater than that of DMXBA on α7 receptors from both mammalian species. Prior exposure of the receptors to a hydroxyl compound also caused much less residual inhibition (reduced response to ACh 5 mm after washing) in comparison with DMXBA. Whereas 100 pM DMXBA produced nearly 50% residual inhibition, the monohydroxy compounds at this concentration displayed little residual inhibitory effect.

TABLE 2

| | Rat Receptor | | Human Receptor | |
|---|---|---|---|---|
| | | Max. | | Max. |
| Compound | EC₅₀ (Ion) μM | Response | EC₅₀(Ion) μM | Response |
| DMXBA | 2.9 ± 0.08 (1.8) | 059 ± 0.05 | 6.0 ± 2.4 (3.7) | 0.23 ± 0.02 |
| 4-OH-MBA | 1.6 ± 0.02 (0.53) | 0.77 ± 0.02 | 4.01 − 0.8 (1.3) | 0.44 ± 0.03 |
| 2-OH-MBA | 3.3 ± 0.8 (1.1) | 0.82 ± 0.06 | 2.1 ± 0.8 (0.67) | 0.53 ± 0.05 |
| 2,4-OH-BA | 3.9 ± 0.6 (2.0) | 0.67 ± 0.03 | 3.2 ± 0.5 (1.6) | 0.49 ± 0.02 |

At the rat α₄β₂ receptor, DMXBA was previously reported to have little (<3% of 5 the maximal ACh current) or no agonistic action (de Eiebre et al, Mol Pharmacol, 1995, 41:31-37). Similarly, we found that 0.3 and 3 μM concentrations of the four compounds tested caused barely measurable agonistic effects on human α₄β₂ receptors. [xposure to these metabolites or DMXBA caused some residual inhibition, as measured after 5 mm of washing the oocytes with saline. Their ability to inhibit the human α₄β₂ receptor was also limited when 3 μM concentrations were co-applied with ACh. These results indicate that all four compounds are agonists at these receptors.

### Example 4

### Nicotine Receptor Binding Studies

Binding at the α₄β₂ nicotinic acetyicholine receptor can be measured as described in Abood, et al., Biochem. Pharmacol., 1986, 35:4199, or in Boksa, et al, Eur. J Pharmacol., 1987, 139:323. In essence, washed rat cerebral cortex membranes suspended in ice-cold saline (pH 7.4) were incubated with [³H]-N-methylcarbamyl choline (³H—MCC) for 60 minutes before being washed free of unbound radioligand with two rapid washes of the saline solution, using vacuum filtration. The glass filters used to collect the membranes for scintillation counting were pre-equilibrated with polyethyleneimine to reduce ligand binding by the filters.

Atropine sulfate at 12 μM was used to block muscarinic binding sites. Non-specific binding was assessed in the presence of 100 μM carbamylcholine. Thus, the K_{d} values of the test compounds were determined by their ability to compete for [³H-acetyl]choline or ³H—MCC binding. The apparent Kₗ values were calculated from the Cheng-Prusoff equation assuming a K_{d} for [³H]—MCC of 5 nM. Some representative compounds of Formula I were tested for their ability to displace [³H]—MCC. Results are indicated in Table 3. The Kₗ values presented in the table are mean values obtained from two separate experiments. Table 3 also shows data for binding of selected compounds of Formula I at the α7 receptor as measured by displacement of iodinated α-bungarotoxin, as described in Example 2. In Table 3, “NA.” means that the data are not available. These results show that the compounds tested [with Kᵢ<5] bind to rat brain synaptosome brain nicotinic receptors with high affinity.

TABLE 3

| Compound | α7 Ki (μM) | α₄β₂ Ki (μM) |
|---|---|---|
| GTS-2 | 0.57 | 0.32 |
| GTS-3 | NA. | >5 |
| GTS-5 | 2.3 | >5 |
| GTS-7 | 0.36 | 0.025 |
| GTS-8 | 1.1 | >5 |
| GTS-13 | 0.05 | 0.17 |
| GTS-15 | NA. | 1.6 |
| GTS-16 | NA. | 0.83 |
| GTS-20 | NA. | 4.0 |
| GTS-21 | 0.13 | 0.12 |
| GTS-23 | NA. | >5 |
| GTS-26 | 0.40 | 0.026 |
| GTS-27 | >10 | 33 |
| GTS-28 | >10 | 3.8 |
| GTS-35 | 0.058 | 0.17 |
| GTS-38 | >10 | 2.0 |
| GTS-39 | NA. | 1.0 |
| GTS-40 | 1.5 | 0.5 |
| GTS-43 | NA. | 0.64 |
| GTS-44 | NA. | 0.80 |
| GTS-45 | NA. | 0.86 |
| GTS-48 | 17 | 2.0 |
| GTS-51 | 0.32 | 1.5 |
| GTS-52 | 0.20 | 0.45 |
| GTS-53 | NA. | 4.5 |
| GTS-54 | N.A. | 1.0 |
| GTS-55 | NA. | 2.5 |
| GTS-56 | NA. | 0.25 |
| GTS-57 | 0.05 | 0.50 |
| GTS-58 | >20 | >5 |
| GTS-60 | 0.08 | 0.13 |
| GTS-63 | >10 | >5 |
| DMACA | 0.07 | 0.40 |

### Example 5

### Evaluation of Healing of Open Ischemic Wounds

Following the method of Canapp et al. (Vet Surg., 2003, 32(6):51 5-23), the effects of topically applied OMXBA (GTS-21), 2-OH-MBA (GTS-51), 2,4-OH—BA (GTS52) and 4-OH-MBA (GTS-62) on healing in ischemic open wounds are evaluated. Forty-eight adult male Sprague-Dawley rats are divided into six groups: topical DMXBA, topical 2-OH-MBA, topical 2,4-OH—BA, topical 4-OH-MBA, topical vehicle (hydroxypropyl-methylcellulose), and no treatment (control). Six-mm-diameter, full-thickness wounds are created within an ischemic bipedicle skin flap on the dorsum of each rat. Each day, for 13 days, wound margins are traced, and the test compound and vehicle groups are treated topically. Tracings are scanned and wound perimeter and area are calculated. On days 6, 10, and 13, selected wounds are biopsied and analyzed for tumor necrosis factor alpha (TNF-alpha) and matrix metalloproteinases (MMP) 2 and 9. A significant decrease in wound area in any of the test compound groups, when compared with the control group on comparable days indicates test compound efficacy. Decreases in TNF-alpha, MMP-2 and MMP-9 in test-compound-treated wounds vs. control wounds also indicate test compound efficacy.

### Example 6

### Reduction of Angiogenesis in Mouse Tumor Model

Following the method of Siemann and Rojiani (Int J. Radiat Oncol Biol Phys., 2002, 54(5):1512-7), C3H/HeJ and NCR/nu-nu mice bearing intramuscular tumors are injected intraperitoneally with GTS-15 (0-150 mg/kg) or GTS-57 (0-150 mg/kg) either alone or in combination or with a vehicle control. Tumor response to treatment is assessed by clonogenic cell survival. The treated mice are assessed for damage to existing neovasculature. Histologic evaluation is also performed to assess dose-dependent morphologic damage of tumor cells within a few hours after drug exposure and after a period of time sufficient to allow for central tumor necrosis and neoplastic cell death as a result of prolonged ischemia. Data are analyzed by appropriate statistical methods to determine the anti-angiogenic or anti-neovascularization effects of the test compounds.

### Example 7

### Measurement of Alpha 7 Receptor Binding Selectivities

After decapitation, washed whole rat brain membranes (200 μg of protein) were prepared according to the method used by Marks and Collins(1982). Displacement of ¹²⁵I-labelled alpha-bungarotoxin (BTX) measured binding to alpha7 receptors; displacement of [³H]-labelled cytisine measured binding to alpha4beta2 receptors. Before use, the washed membranes were resuspended in 500 μl receptor binding assay saline (pH 7.4) consisting of 120 mM NaCl, 5 mM KCl, 2mM CaCl₂, 1 mM MgCl₂ and 50 mM Tris-HCl. [³H]Cytisine (35 Ci/mmole)-binding displacement experiments were performed essentially according to Flores, et al. (1992), except that the incubation time was increased to 4 hr at 0 to 4° C. to ensure equilibrium during the competition binding assay. Binding of ¹²⁵I-BTX (136 Ci/mmole) was performed at 37° C. for 3 h; the saline solution mentioned above also contained 2 mg/ml bovine serum albumin. Nonspecific binding of each radioligand was measured in the presence of 1.0 mM nicotine. After incubation, membranes with bound radioligand were collected on Whatman GF/C glass filber filters presoaked for 45 min in 0.5% polyethylenimine and washed three times with 3.0 ml of ice-cold buffer by vacuum filtration on a harvester (Brandel, Gaithersburg, Md.). Bound [³H] cytosine was measured in a liquid scintillation counter, whereas [¹²⁵]BTX was measured with use of a Biogamma counter (both from Beckman coulter). Binding studies were analyzed using Prism software (GraphPad Software Inc., San Diego, Calif.). All Ki values were calculated from the Cheng-Prusoff equation, using a Kd value for each radioligand that had been experimentally determined under conditions identical with those of the displacement experiments. The alpha7 binding selectivity of each compound shown in Table 1 was estimated by dividing the Ki for aplha4beta2 binding by the Ki for alpha7 binding. The alpha7 binding selectivity of each compound was calculated by dividing the Ki for alpha4beta2 binding by the Ki for alpha7 binding.

All publications and patent applications mentioned in this specification are herein incorporated by reference to the same extent as if each individual publication or patent application were specifically and individually indicated to be incorporated by reference.

The invention now being fully described, it will be apparent to one of ordinary skill in the art that many changes and modifications can be made thereto without departing from the spirit or scope of the appended claims.

Other Embodiments

This description has been by way of example of how the compositions and methods of the invention can be made and carried out. Various details may be modified in arriving at the other detailed embodiments, and many of these embodiments will come within the scope of the invention. Therefore, to apprise the public of the scope of the invention and the embodiments covered by the invention, the following claims are made.

1. A method of stimulating angiogenesis in a mammal, the method comprising administering to a mammal an anabaseine agonist in an amount effective to stimulate angiogenesis.

2. The method of claim 1, wherein the anabaseine agonist is a compound of the formula: 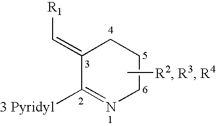
or a pharmaceutically acceptable salt thereof, wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, methyl, propyl, ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl and/or,
wherein R³ and R⁴ are each selected from hydrogen, methyl, methoxy, methyl, propyl, ethyl, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro.

3. The method of claim 2, wherein the R² at position 4, R³ at position 5 and R⁴ at position 6 on the terahydropyridyl ring are selected from the group consisting of methyl, propyl and ethyl.

4. The method of claim 3, wherein the R² at position 4, R³ at position 5 and R⁴ at position 6 are substituted at each position with one or more of methyl, propyl and ethyl.

5. The method of claim 3, wherein the methyl, propyl and ethyl groups are in (S)⁻ or (R)⁻ enantiomeric form.

6. The method of claim 1, wherein the anabaseine agonist is a compound of the formula: 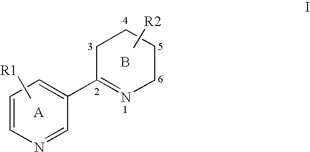
or a pharmaceutically acceptable salt thereof, wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, propyl, ethyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or 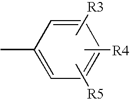
wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ,
wherein Z is 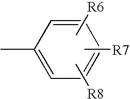
wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; and,
R¹—R⁶ are in an (S)⁻ enantiomeric or (R)⁻ enantiomeric form.

7. The method of claim 6, wherein said compound is selected from the group consisting of
GTS-2, 3-(4-Methoxybenzylidene)anabaseine,
GTS-3, 3-(4-Nitrobenzylidene)anabaseine,
GTS-5, 3-(4-Cyanobenzylidene)anabaseine,
GTS-7, 3-(4-Hydroxybenzylidene)anabaseine,
GTS-8, 3-(4-Chlorobenzylidene)anabaseine,
GTS-13,3-(4-Aminobenzylidene)anabaseine,
GTS-15, 3-(4-Dimethylaminopropoxy-benzylidene)anabaseine,
GTS-16, 3-(2-Methoxybenzylidene)anabaseine,
GTS-20, 3-(3-Methoxybenzylidene)anabaseine,
GTS-21, DMXBA, 3-(2,4-Dimethoxybenzylidene)anabaseine,
GTS-23, 3-(3-Methoxy-4-hydroxybenzylidene)anabaseine,
GTS-26, 6′-Methylanabaseine,
GTS-27, 2′-Methylanabaseine,
GTS-28, 4′-Methylanabaseine,
GTS-35, 3-(2,4,6-Trimethoxybenzylidene)anabaseine,
GTS-38, 3-(2,4-Dichlorobenzylidene)anabaseine,
GTS-39, 3-(2,4-Dimethylbenzylidene)anabaseine,
GTS-40, 3-(2,46,-Trimethylbenzylidene)anabaseine,
GTS-43, 3-(2-Furylidene)anabaseine,
GTS-44, 3-(2-Furylpropenylidene)anabaseine,
GTS-45, 3(3-Furylidene)anabaseine,
GTS-48, 3-(4-Methylbenzylidene)anabaseine,
GTS-51, 3-(2-Hydroxy-4-methoxybenzylidene)anabaseine,
GTS-52, 3-(2,4-Dihydroxybenzylidene)anabaseine,
GTS-53, 3-(2,4-Dipropoxybenzylidene)anabaseine,
GTS-54, 3-(2,4-Diisopropoxybenzylidene)anabaseine,
GTS-55, 3-(2,4-Dipentoxybenzylidene)anabaseine,
GTS-56, 3-(2-Hydroxy-4-pentoxybenzylidene)anabaseine,
GTS-57, 6′-Methyl-3-(2,4-dimethoxybenzylidene)anabaseine,
GTS-58, 1-Methyl-3-(2,4-djmethoxybenzylidene)anabaseine trifluoroacetate,
GTS-60, 5′-Methylanabaseine,
GTS-62, 3-(2-Methoxy-4-hydroxybenzylidene)anabaseine,
GTS-63, 2-Phenyl-3-(2,4-dimethoxybenzylidene)-4,5,6-trihydropyridine; and DMACA, 3-(4-Dimethylaminocinnamylidene)anabaseine, and
GTS-83

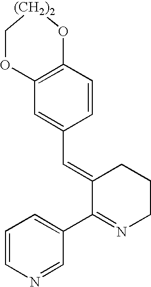

8. The method of claim 7, wherein said compound is GTS-21.

9. The method of claim 1, wherein said compound is selected from the group consisting of GTS-51, GTS-52 and GTS-62.

10. The method of claim 1, wherein said administering is intravenous, intra-arterial, or intra-pericardial.

11. The method of claim 1, wherein said administering is intramuscular or at a local site.

12. The method of claim 1, wherein said administering is systemic.

13. The method of claim 1, wherein said administering is by inhalation.

14. The method of claim 1, wherein said administering is topical or transdermal.

15. The method of claim 1, wherein said administering is for stimulation of angiogenesis after surgery.

16. The method of claim 15, wherein said administering is at a site of an anastomosis, suture line, or surgical wound.

17. The method of claim 1, wherein said administering is by application of the anabaseine agonist to a region in or adjacent ischemic tissue.

18. The method of claim 1, the method further comprising administering an agent that enhances synthesis or activity of nitric oxide.

19. The method of claim 18, wherein the agent is selected from the group consisting of a nitric oxide substrate, an antioxidant, and a nitric oxide synthase cofactor.

20. The method of claim 19, wherein the agent is selected from the group consisting of L-arginine, L-lysine, tocopherol, ascorbic acid, ubiquinone, superoxide dismutase, tetrahydrobiopterin, and sepiapterin.

21. The method of claim 1, the method further comprising administering an amount of an agent that enhances synthesis or activity of prostacyclin.

22. The method of claim 21, wherein the agent is selected from the group consisting of eicosapentanoic acid, docosoahexanoic acid, prostaglandin El, and a prostaglandin El analogue.

23. The method of claim 1, wherein said administering is effective to stimulate angiogenesis in or around a wound.

24. The method of claim 1, wherein said administering is effective to stimulate angiogenesis in or around an ulcer.

25. The method of claim 1, wherein said administering is effective to stimulate angiogenesis in or around a skin graft.

26. The method of claim 1, wherein said administering is effective to stimulate angiogenesis in or around a transplanted tissue.

27. The method of claim 1, wherein said administering is effective to stimulate angiogenesis at a reattached limb.

28. A method of inhibiting angiogenesis in a mammal, the method comprising administering to a mammal an anabaseine antagonist in an amount effective to inhibit angiogenesis.

29. The method of claim 24, wherein the anabaseine antagonist a compound of the formula: 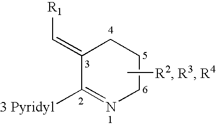
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, methyl, propyl, ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl and/or,
wherein R³ and R⁴ are each selected from hydrogen, methyl, methoxy, methyl, propyl, ethyl, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro.

30. The method of claim 29, wherein the R² at position 4, R³ at position 5 and R⁴ at position 6 on the terahydropyridyl ring are selected from the group consisting of methyl, propyl and ethyl.

31. The method of claim 30, wherein the R² at position 4, R³ at position 5 and R⁴ at position 6 are substituted at each position with one or more of methyl, propyl and ethyl.

32. The method of claim 30, wherein the methyl, propyl and ethyl groups are in (S)⁻ or (R)⁻ enantiomeric form.

33. The method of claim 24, wherein the anabaseine antagonist a compound of the formula: 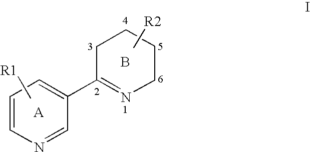
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, propyl, ethyl, hydrogen, (S,R)-methyl, S— or R-methyl, (S,R)-propyl, S— or R-propyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or 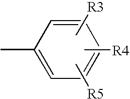
wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is 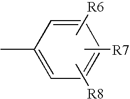
wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; and,
R¹—R⁶ are in an (S)⁻ enantiomeric or (R)⁻ enantiomeric form.

34. The method of claim 33, wherein said compound is selected from the group consisting of GTS-2, 3-(4-Methoxybenzylidene)anabaseine,
GTS-3, 3-(4-Nitrobenzylidene)anabaseine,
GTS-5, 3-(4-Cyanobenzylidene)anabaseine,
GTS-7, 3-(4-Hydroxybenzylidene)anabaseine,
GTS-8, 3-(4-Chlorobenzylidene)anabaseine,
GTS-13,3-(4-Aminobenzylidene)anabaseine,
GTS-15, 3-(4-Dimethylaminopropoxy-benzylidene)anabaseine,
GTS-16, 3-(2-Methoxybenzylidene)anabaseine,
GTS-20, 3-(3-Methoxybenzylidene)anabaseine,
GTS-21, DMXBA, 3-(2,4-Dimethoxybenzylidene)anabaseine,
GTS-23, 3-(3-Methoxy-4-hydroxybenzylidene)anabaseine,
GTS-26, 6′-Methylanabaseine,
GTS-27, 2′-Methylanabaseine,
GTS-28, 4′-Methylanabaseine,
GTS-35, 3-(2,4,6-Trimethoxybenzylidene)anabaseine,
GTS-38, 3-(2,4-Dichlorobenzylidene)anabaseine,
GTS-39, 3-(2,4-Dimethylbenzylidene)anabaseine,
GTS-40, 3-(2,46,-Trimethylbenzylidene)anabaseine,
GTS-43, 3-(2-Furylidene)anabaseine,
GTS-44, 3-(2-Furylpropenylidene)anabaseine,
GTS-45, 3(3-Furylidene)anabaseine,
GTS-48, 3-(4-Methylbenzylidene)anabaseine,
GTS-51, 3-(2-Hydroxy-4-methoxybenzylidene)anabaseine,
GTS-52, 3-(2,4-Dihydroxybenzylidene)anabaseine,
GTS-53, 3-(2,4-Dipropoxybenzylidene)anabaseine,
GTS-54, 3-(2,4-Diisopropoxybenzylidene)anabaseine,
GTS-55, 3-(2,4-Dipentoxybenzylidene)anabaseine,
GTS-56, 3-(2-Hydroxy-4-pentoxybenzylidene)anabaseine,
GTS-57, 6′-Methyl-3-(2,4-dimethoxybenzylidene)anabaseine,
GTS-58, 1-Methyl-3-(2,4-djmethoxybenzylidene)anabaseine trifluoroacetate,
GTS-60, 5′-Methylanabaseine,
GTS-62, 3-(2-Methoxy-4-hydroxybenzylidene)anabaseine,
GTS-63, 2-Phenyl-3-(2,4-dimethoxybenzylidene)-4,5,6-trihydropyridine; and DMACA, 3-(4-Dimethylaminocinnamylidene)anabaseine.

35. The method of claim 34, wherein said compound is selected from the group consisting of GTS-15 and GTS-57.

36. The method of claim 33, wherein said administering is by a route selected from the group consisting of intravenous, in or around a solid tumor, systemic, intra-arterial, and topical.

37. A method of treating a disorder associated with pathological angiogenesis, the method comprising administering to a mammal an anabaseine antagonist in an amount effective to reduce pathological angiogenesis.

38. The method of claim 37, wherein the anabaseine antagonist is a compound of the formula: 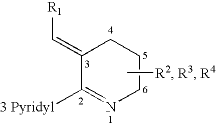
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, methyl, propyl, ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl and/or,
wherein R³ and R⁴ are each selected from hydrogen, methyl, methoxy, methyl, propyl, ethyl, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro.

39. The method of claim 38, wherein the R² at position 4, R³ at position 5 and R⁴ at position 6 on the terahydropyridyl ring are selected from the group consisting of methyl, propyl and ethyl.

40. The method of claim 39, wherein the R² at position 4, R³ at position 5 and R⁴ at position 6 are substituted at each position with one or more of methyl, propyl and ethyl.

41. The method of claim 39, wherein the methyl, propyl and ethyl groups are in (S)⁻ or (R)⁻ enantiomeric form.

42. The method of claim 37, wherein the anabaseine antagonist a compound of the formula: 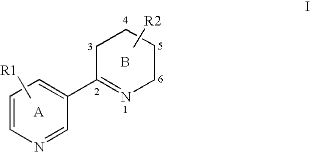
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, ethyl, propyl, hydrogen, (S,R)-methyl, S— or R-methyl, (S,R)-propyl, S— or R-propyl, or ═CH—X,
wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or 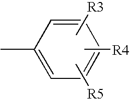
wherein R³, R⁴, and R⁵ are each selected from hydrogen, methyl, propyl, ethyl, methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is 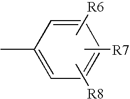
wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; and,
R¹—R⁶ are in an (S)⁻ enantiomeric or (R)⁻ enantiomeric form.

43. The method of claim 42, wherein said compound is selected from the group consisting of
GTS-2, 3-(4-Methoxybenzylidene)anabaseine,
GTS-3, 3-(4-Nitrobenzylidene)anabaseine,
GTS-5, 3-(4-Cyanobenzylidene)anabaseine,
GTS-7, 3-(4-Hydroxybenzylidene)anabaseine,
GTS-8, 3-(4-Chlorobenzylidene)anabaseine,
GTS-13,3-(4-Aminobenzylidene)anabaseine,
GTS-15, 3-(4-Dimethylaminopropoxy-benzylidene)anabaseine,
GTS-16, 3-(2-Methoxybenzylidene)anabaseine,
GTS-20, 3-(3-Methoxybenzylidene)anabaseine,
GTS-21, DMXBA, 3-(2,4-Dimethoxybenzylidene)anabaseine,
GTS-23, 3-(3-Methoxy-4-hydroxybenzylidene)anabaseine,
GTS-26, 6′-Methylanabaseine,
GTS-27, 2′-Methylanabaseine,
GTS-28, 4′-Methylanabaseine,
GTS-35, 3-(2,4,6-Trimethoxybenzylidene)anabaseine,
GTS-38, 3-(2,4-Dichlorobenzylidene)anabaseine,
GTS-39, 3-(2,4-Dimethylbenzylidene)anabaseine,
GTS-40, 3-(2,46,-Trimethylbenzylidene)anabaseine,
GTS-43, 3-(2-Furylidene)anabaseine,
GTS-44, 3-(2-Furylpropenylidene)anabaseine,
GTS-45, 3(3-Furylidene)anabaseine,
GTS-48, 3-(4-Methylbenzylidene)anabaseine,
GTS-51, 3-(2-Hydroxy-4-methoxybenzylidene)anabaseine,
GTS-52, 3-(2,4-Dihydroxybenzylidene)anabaseine,
GTS-53, 3-(2,4-Dipropoxybenzylidene)anabaseine,
GTS-54, 3-(2,4-Diisopropoxybenzylidene)anabaseine,
GTS-55, 3-(2,4-Dipentoxybenzylidene)anabaseine,
GTS-56, 3-(2-Hydroxy-4-pentoxybenzylidene)anabaseine,
GTS-57, 6′-Methyl-3-(2,4-dimethoxybenzylidene)anabaseine,
GTS-58, 1-Methyl-3-(2,4-djmethoxybenzylidene)anabaseine trifluoroacetate,
GTS-60, 5′-Methylanabaseine,
GTS-62, 3-(2-Methoxy-4-hydroxybenzylidene)anabaseine,
GTS-63, 2-Phenyl-3-(2,4-dimethoxybenzylidene)-4,5,6-trihydropyridine; and DMACA, 3-(4-Dimethylaminocinnamylidene)anabaseine, and
GTS-83

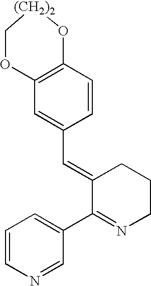

44. The method of claim 43, wherein said compound is selected from the group consisting of GTS-15 and GTS-57.

45. The method of claim 37, wherein said administering is by a route selected from the group consisting of intravenous, in or around a solid tumor, systemic, intra-arterial, and topical.

46. The method of claim 37, further comprising administering a second angiogenesis inhibitor.

47. A method of inhibiting tumor growth in a mammal, the method comprising administering to a mammal having a tumor an anabaseine antagonist in an amount effective to reduce angiogenesis, wherein said administering is peritumoral, and wherein a reduction in angiogenesis inhibits tumor growth.

48. The method according to claim 47, further comprising administering an anti-tumor chemotherapeutic agent.

49. A method of inhibiting abnormal fibrovascular growth in a mammal, the method comprising administering to a mammal having abnormal fibrovascular growth an anabaseine antagonist in an amount effective to reduce abnormal fibrovascular growth in the mammal.

50. The method of claim 49, wherein the abnormal fibrovascular growth is associated with inflammatory arthritis.

51. A method of inhibiting a proliferative retinopathy in a mammal, the method comprising administering to a mammal having a tumor an anabaseine antagonist in an amount effective to reduce the proliferative retinopathy in the mammal.

52. The method according claim 51, wherein the proliferative retinopathy occurs as a result of diabetes in the mammal.

53. A method of inhibiting pathological neovascularization associated with a tumor, the method comprising administering to a mammal having a tumor an anabaseine antagonist in an amount effective to reduce the tumor-associated pathological neovascularization in the mammal.

54. An anabaseine compound of the formula: 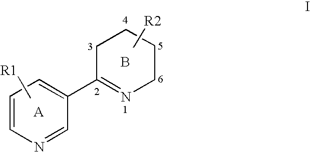
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, or ═CH—X,
wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl or 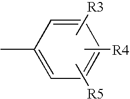
wherein R³, R⁴, and R⁵ are each selected from hydrogen, methyl, propyl, ethyl,methoxy, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro; or R² is ═CHCH═CHZ, wherein Z is 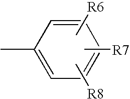
wherein R⁶, R⁷, and R⁸ are selected from the group consisting of hydrogen, methyl, propyl, ethyl, methoxy, cyano-, phenoxy, phenyl, pyridyl or benzyl, C₁-C₄ alkyl optionally substituted with N,N-dialkylamino having 1 to 4 carbons in each of the alkyls, C₁-C₆ alkoxy optionally substituted with N,N-dialkylamino having 1 to 4 carbons in each of the alkyls, carboalkoxy having 1 to 4 carbons in the alkoxy, amino, acetylamino having 1 to 4 carbons in the acyl, cyano, N,N-dialkylamino having 1 to 4 carbons in each of the alkyls, halo, hydroxyl, and nitro; and,
R¹—R⁶ are in an (S)⁻ enantiomeric or (R)⁻ enantiomeric form.

55. The compound of claim 54, wherein R² at position 4, R³ at position 5 and R⁴ at position 6 of a terahydropyridyl ring are selected from the group consisting of: methyl, propyl and ethyl.

56. The compound of claim 55, wherein substitutions :R² at position 4, R³ at position 5 and R⁴ at position 6 on the terahydropyridyl ring are at least one of: methyl, propyl, and ethyl, groups in (S)⁻ enantiomeric form.

57. The compound of claim 55, wherein substitutions :R² at position 4, R³ at position 5 and R⁴ at position 6 on the terahydropyridyl ring are at least one of: methyl, propyl, and ethyl, groups in (R)⁻ enantiomeric form.

58. The compound of claim 54, wherein said compound is selected from the group consisting of GTS-2, 3-(4-Methoxybenzylidene)anabaseine,
GTS-3, 3-(4-Nitrobenzylidene)anabaseine,
GTS-5, 3-(4-Cyanobenzylidene)anabaseine,
GTS-7, 3-(4-Hydroxybenzylidene)anabaseine,
GTS-8, 3-(4-Chlorobenzylidene)anabaseine,
GTS-13,3-(4-Aminobenzylidene)anabaseine,
GTS-15, 3-(4-Dimethylaminopropoxy-benzylidene)anabaseine,
GTS-16, 3-(2-Methoxybenzylidene)anabaseine,
GTS-20, 3-(3-Methoxybenzylidene)anabaseine,
GTS-21, DMXBA, 3-(2,4-Dimethoxybenzylidene)anabaseine,
GTS-23, 3-(3-Methoxy-4-hydroxybenzylidene)anabaseine,
GTS-26, 6′-Methylanabaseine,
GTS-27, 2′-Methylanabaseine,
GTS-28, 4′-Methylanabaseine,
GTS-35, 3-(2,4,6-Trimethoxybenzylidene)anabaseine,
GTS-38, 3-(2,4-Dichlorobenzylidene)anabaseine,
GTS-39, 3-(2,4-Dimethylbenzylidene)anabaseine,
GTS-40, 3-(2,46,-Trimethylbenzylidene)anabaseine,
GTS-43, 3-(2-Furylidene)anabaseine,
GTS-44, 3-(2-Furylpropenylidene)anabaseine,
GTS-45, 3(3-Furylidene)anabaseine,
GTS-48, 3-(4-Methylbenzylidene)anabaseine,
GTS-51, 3-(2-Hydroxy-4-methoxybenzylidene)anabaseine,
GTS-52, 3-(2,4-Dihydroxybenzylidene)anabaseine,
GTS-53, 3-(2,4-Dipropoxybenzylidene)anabaseine,
GTS-54, 3-(2,4-Diisopropoxybenzylidene)anabaseine,
GTS-55, 3-(2,4-Dipentoxybenzylidene)anabaseine,
GTS-56, 3-(2-Hydroxy-4-pentoxybenzylidene)anabaseine,
GTS-57, 6′-Methyl-3-(2,4-dimethoxybenzylidene)anabaseine,
GTS-58, 1-Methyl-3-(2,4-djmethoxybenzylidene)anabaseine trifluoroacetate,
GTS-60, 5′-Methylanabaseine,
GTS-62, 3-(2-Methoxy-4-hydroxybenzylidene)anabaseine,
GTS-63, 2-Phenyl-3-(2,4-dimethoxybenzylidene)-4,5,6-trihydropyridine, DMACA, 3-(4-Dimethylaminocinnamylidene)anabaseine, and
GTS-83

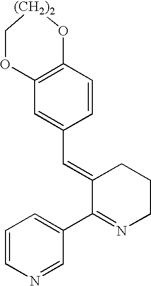

59. An anabaseine compound of the formula: 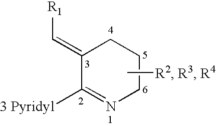
or a pharmaceutically acceptable salt thereof; wherein R¹ is hydrogen acetoxy, acetamido, amino, dimethylcarbamate, dimethylaminopropoxy, hydroxyl, methoxy, methyl, propyl, ethyl, isopropoxy, trifluromethoxy or thiomethoxy or C₁-C₄ alkyl; and R² is hydrogen, methyl, hydrogen, (S,R)-methyl, S— or R-methyl, (S,R)-propyl, S— or R-propyl, methyl, propyl, ethyl, or ═CH—X, wherein X is napthyl optionally substituted by N,N-dialkylamino having 1 to 4 carbons in each of the alkyls, styryl optionally substituted by N,N-dialkylamino having 1 to 4 carbons in each of the alkyls, furyl, furylacrolyl and/or,
wherein R³ and R⁴ are each selected from hydrogen, methyl, methoxy, methyl, propyl, ethyl, cyano-, phenoxy, phenyl, pyridyl or benzyl C₁-C₄ alkyl, C₁-C₆ alkoxy optionally substituted by N,N-dialkylamino having 1 to 4 carbons in each of the alkyls, amino, cyano, N,N-dialkylamino having 1 to 4 carbons in each of the alkyls, halo, hydroxyl, and nitro.

60. The compound of claim 59, wherein the R² at position 4, R³ at position 5 and R⁴ at position 6 on the terahydropyridyl ring are selected from the group consisting of methyl, propyl and ethyl.

61. The compound of claim 60, wherein the R² at position 4, R³ at position 5 and R⁴ at position 6 are substituted at each position with one or more of methyl, propyl and ethyl.

62. The compound of claim 59, wherein substitutions :R² at position 4, R³ at position 5 and R⁴ at position 6 on the terahydropyridyl ring are at least one of: methyl, propyl, and ethyl, groups in (S)⁻ enantiomeric form.

63. The compound of claim 59, wherein substitutions :R² at position 4, R³ at position 5 and R⁴ at position 6 on the terahydropyridyl ring are at least one of: methyl, propyl, and ethyl, groups in (R)⁻ enantiomeric form.
